SEQUENCE LISTING (1 sequence)

SEQ ID NO: 1 - DNA, 12 bp, synthetic construct
  source 1..12: mol_type = other DNA
tatggtcgtcga

(19) United States
(12) Patent Application Publication
(10) Pub. No.: US 2024/0386998 A1
(43) Pub. Date: Nov. 21, 2024

(54) METHODS AND SYSTEMS FOR OBTAINING AND PROCESSING SEQUENCING DATA

(71) Applicant: Ultima Genomics, Inc., Fremont, CA (US)

(72) Inventors: Simchon FAIGLER, Bet Izhak (IL); Eyal NEISTEIN, Herzliya (IL); Mark PRATT, Bozeman, MT (US)

(21) Appl. No.: 18/426,104

(22) Filed: Jan. 29, 2024

Related U.S. Application Data
(63) Continuation of application No. PCT/US2022/074349, filed on Jul. 29, 2022.
(60) Provisional application No. 63/266,397, filed on Jan. 4, 2022.
(60) Provisional application No. 63/203,791, filed on Jul. 30, 2021.

(51) Int. Cl. G16B 30/00; G06T 5/20; G06T 5/50; G06T 7/00; G06T 7/194; G06V 20/69; G06V 20/70

(52) U.S. Cl. CPC G16B 30/00; G06T 5/20; G06T 5/50; G06T 7/0012; G06T 7/194; G06V 20/698; G06V 20/70; G06T 2207/20224; G06T 2207/30072

(57) ABSTRACT

The present disclosure relates generally to sequencing techniques, and more specifically to methods, systems, devices, and non-transitory computer-readable storage media for processing images of biological samples (e.g., to obtain sequencing data). An exemplary method of determining nucleic acid sequences of a plurality of sequencing colonies comprises: obtaining an input image of a surface, wherein the plurality of sequencing colonies are attached to the surface; detecting a set of sequencing colonies of the plurality of sequencing colonies in the input image; executing in parallel, using a graphics processor, a plurality of iterative processes to obtain signal amplitudes for the detected set of sequencing colonies, wherein each iterative process corresponds to a respective detected sequencing colony in the set; and determining, at least partially based on the signal amplitudes for the detected set of sequencing colonies, portions of nucleic acid sequences of the plurality of sequencing colonies.

### CROSS-REFERENCE TO RELATED APPLICATIONS

This application is a continuation of PCT Application No. PCT/US2022/074349, filed internationally on Jul. 29, 2022, which claims the priority benefit of U.S. Provisional Patent Application Ser. No. 63/203,791, filed on Jul. 30, 2021, and to U.S. Provisional Patent Application Ser. No. 63/266,397, filed on Jan. 4, 2022, the contents of each of which are incorporated herein by reference in their entirety.

### REFERENCE TO A SEQUENCE LISTING

The contents of the electronic sequence listing (165272001201SEQLIST.xml; Size: 1,947 bytes; and Date of Creation: Jan. 29, 2024) is herein incorporated by reference in its entirety.

### FIELD OF INVENTION

The present disclosure relates generally to sequencing techniques, and more specifically to methods, systems, devices, and non-transitory computer-readable storage media for processing images of biological samples (e.g., to obtain sequencing data).

### BACKGROUND

A sequencing system can operate by detecting signals (e.g., fluorescence signals) from biological samples and using the detected signals to derive sequencing data (e.g., nucleic acid sequences). Specifically, the biological samples can be captured in image data, and the image data can be analyzed to detect one or more properties of the signals (e.g., intensity) to derive sequencing data.

Conventional techniques for detecting signal intensities of one or more objects captured in a given image typically involve identifying a peak amplitude associated with each object in the image. This simplistic approach can be inaccurate, especially when processing images of biological samples such as images captured during a flow sequencing method. For example, conventional techniques can produce inaccurate results due to failure to account for signal interference or crosstalk from neighboring objects.

Further, the conventional approach, which typically relies on generic computer processors, is computationally expensive when processing image data generated during flow sequencing. During flow sequencing, a large volume of high-definition images can be generated at a high rate. These images need to be processed at a high rate (e.g., thousands, tens of thousands, hundreds of thousands of images per second). The conventional approach relying on generic processors would not be able to process the images at such a high rate to support timely and efficient performance of the flow sequencing method. Furthermore, the conventional approach, which typically relies on linear or serial processing to process image data leads to an inefficient use of computer processing power and computer memory, again failing to support timely and efficient performance of the flow sequencing method.

### BRIEF SUMMARY

An exemplary method of determining nucleic acid sequences of a plurality of sequencing colonies comprises: obtaining an input image of a surface, wherein the plurality of sequencing colonies are attached to the surface; detecting a set of sequencing colonies of the plurality of sequencing colonies in the input image; executing in parallel, using a graphics processor, a plurality of iterative processes to obtain signal amplitudes for the detected set of sequencing colonies, wherein each iterative process corresponds to a respective detected sequencing colony in the set, and wherein each iterative process comprises: (a) obtaining amplitude, location, and profile estimates of one or more neighboring sequencing colonies to the respective sequencing colony; (b) calculating, using the graphics processor, a crosstalk value for the respective sequencing colony based on the amplitude, location, and profile estimates of the one or more neighboring sequencing colonies; (c) subtracting, using the graphics processor, the crosstalk value and a colony-specific background to obtain a current amplitude estimate of the respective sequencing colony; (d) performing a next iteration of (a)-(c) for a predetermined number of times or until a condition is met; and determining, at least partially based on the signal amplitudes for the detected set of sequencing colonies, portions of nucleic acid sequences of the plurality of sequencing colonies.

In some embodiments, each iterative process further comprises: determining, using the graphics processor, a current location estimate of the respective sequencing colony. In some embodiments, each iterative process further comprises: determining, using the graphics processor, one or more current profile properties of the respective sequencing colony.

In some embodiments, the predetermined number of times is between 5-7 times.

In some embodiments, the input image is a first input image corresponding to a first flow step, the obtained signal amplitudes correspond to the first flow step, and the method further comprises: obtaining a second input image corresponding to a second flow step; and obtaining signal amplitudes corresponding to the second flow step.

In some embodiments, the method further comprises identifying, based on the signal amplitudes corresponding to the first flow step and the second flow step, the nucleic acid sequences of the plurality of sequencing colonies.

In some embodiments, the plurality of sequencing colonies is attached to a plurality of beads attached to the surface.

In some embodiments, the method further comprises: capturing the input image of the surface.

In some embodiments, the method further comprises: combining the plurality of sequencing colonies with nucleotides before capturing the input image, wherein at least a portion of the nucleotides are labeled.

In some embodiments, detecting the set of sequencing colonies comprises: applying one or more filters to the input image. In some embodiments, the one or more filters comprise a Gaussian filter. In some embodiments, the Gaussian filter is based on a known profile of a standard bead attached to the surface. In some embodiments, the known profile includes a shape, a size, or a full-width at half-maximum value of the standard bead. In some embodiments, the one or more filters comprise a low-pass filter and/or a high-pass filter.

In some embodiments, the method further comprises obtaining, based on a global background value, a binary image having a plurality of pixel values.

In some embodiments, the method further comprises grouping, based on the plurality of pixel values, pixels of the binary image into the detected set of sequencing colonies.

In some embodiments, the method further comprises determining a center pixel for each of the detected set of sequencing colonies.

In some embodiments, the method further comprises determining an initial location for each of the detected set of sequencing colonies. In some embodiments, the initial location is a sub-pixel location. In some embodiments, the determination comprises a center of mass estimation.

In some embodiments, the method further comprises: executing in parallel, using the graphics processor, a plurality of processes, each process corresponding to determining a respective sub-pixel location of a respective sequencing colony of the detected set of sequencing colonies.

In some embodiments, the method further comprises: registering a center patch of the input image and a center patch of a reference image to obtain a horizontal shift and a vertical shift of the input image with respect to the reference image. In some embodiments, the reference image is an image in which all captured sequencing colonies emit signals over a predefined threshold. In some embodiments, the registering comprises: generating a first synthetic image corresponding to the center patch of the input image; generating a second synthetic image corresponding to the center patch of the reference image; and correlating the first synthetic image with the second synthetic image.

In some embodiments, each sequencing colony in the center patch of the input image is represented by the same Gaussian profile in the first synthetic image. In some embodiments, each sequencing colony in the center patch of the reference image is represented by the same Gaussian profile in the second synthetic image.

In some embodiments, correlating the first synthetic image with the second synthetic image comprises performing a two-dimensional cross correlation using Fourier transform.

In some embodiments, the method further comprises generating an affine transformation between the reference image and the input image. In some embodiments, the method further comprises iteratively refining one or more coefficients of the affine transformation.

In some embodiments, the method further comprises: in each iteration: applying the affine transformation to the reference image; pairing one or more sequencing colonies in the input image with one or more transformed sequencing colonies in the reference image; and randomly selecting a number of paired sequencing colonies to refine the one or more coefficients of the affine transformation.

In some embodiments, the method further comprises dividing the input image into a plurality of sub-images; identifying, for each sub-image of the plurality of sub-images, a group of pixels in the respective sub-image based on pixel-specific amplitude information; extending, for each sub-image, the respective group of pixels; calculating, for each sub-image, a local background value based on the extended respective group of pixels; and generating a background map based on local background values of the plurality of sub-images.

In some embodiments, the method further comprises: applying a mean filter to the background map.

In some embodiments, the method further comprises deriving a colony-specific background for each detected sequencing colony of the detected set of sequencing colonies by bi-linear interpolation of the background map.

In some embodiments, the method further comprises deriving a global background value based on a median of all extended groups of pixels for the plurality of sub-images.

In some embodiments, the one or more current profile properties include a current full width at half maximum (“FWHM”) estimate, a pseudo-Voigt Lorentzian weight (tail) parameter, or parameters of an elliptic model. In some embodiments, the one or more current profile properties are determined based on an FWHM map.

In some embodiments, the surface is part of a substrate.

In some embodiments, the method further comprises capturing an arc-shaped or ring-shaped image of the surface.

In some embodiments, the method further comprises dividing the captured image into a plurality of image tiles, wherein the input image is one image tile of the plurality of image tiles.

In some embodiments, the method further comprises: executing in parallel, using the graphics processor, a plurality of processes, each process corresponding to a respective image tile of the plurality of image tiles.

In some embodiments, the method further comprises detecting a plurality of sequencing colonies in a reference image; generating a simulated image based on the plurality of detected sequencing colonies in the reference image; subtracting the simulated image from the reference image to obtain a residual image; and detecting one or more additional sequencing colonies based on the residual image.

An exemplary system of determining nucleic acid sequences of a plurality of sequencing colonies comprises: one or more processors; a memory; and one or more programs, wherein the one or more programs are stored in the memory and configured to be executed by the one or more processors, the one or more programs including instructions for obtaining an input image of a surface, wherein the plurality of sequencing colonies are attached to the surface; detecting a set of sequencing colonies of the plurality of sequencing colonies in the input image; executing in parallel, using a graphics processor, a plurality of iterative processes to obtain signal amplitudes for the detected set of sequencing colonies, wherein each iterative process corresponds to a respective detected sequencing colony in the set, and wherein each iterative process comprises: (a) obtaining amplitude, location, and profile estimates of one or more neighboring sequencing colonies to the respective sequencing colony; (b) calculating, using the graphics processor, a crosstalk value for the respective sequencing colony based on the amplitude, location, and profile estimates of the one or more neighboring sequencing colonies; (c) subtracting, using the graphics processor, the crosstalk value and a colony-specific background to obtain a current amplitude estimate of the respective sequencing colony; (d) performing a next iteration of (a)-(c) for a predetermined number of times or until a condition is met; and determining, at least partially based on the signal amplitudes for the detected set of sequencing colonies, portions of nucleic acid sequences of the plurality of sequencing colonies.

In some embodiments, each iterative process further comprises: determining, using the graphics processor, a current location estimate of the respective sequencing colony.

In some embodiments, each iterative process further comprises: determining, using the graphics processor, one or more current profile properties of the respective sequencing colony.

In some embodiments, the predetermined number of times is between 5-7 times.

In some embodiments, the input image is a first input image corresponding to a first flow step, the obtained signal amplitudes correspond to the first flow step, and the method further comprises: obtaining a second input image corresponding to a second flow step; and obtaining signal amplitudes corresponding to the second flow step.

In some embodiments, the one or more programs further include instructions for: identifying, based on the signal amplitudes corresponding to the first flow step and the second flow step, the nucleic acid sequences of the plurality of sequencing colonies.

In some embodiments, the plurality of sequencing colonies are attached to a plurality of beads attached to the surface.

In some embodiments, the one or more programs further include instructions for: capturing the input image of the surface.

In some embodiments, the one or more programs further include instructions for: combining the plurality of sequencing colonies with nucleotides before capturing the input image, wherein at least a portion of the nucleotides are labeled.

In some embodiments, detecting the set of sequencing colonies comprises: applying one or more filters to the input image.

In some embodiments, the one or more filters comprise a Gaussian filter.

In some embodiments, the Gaussian filter is based on a known profile of a standard bead attached to the surface.

In some embodiments, the known profile includes a shape, a size, or a full-width at half-maximum value of the standard bead.

In some embodiments, the one or more filters comprise a low-pass filter and/or a high-pass filter.

In some embodiments, the one or more programs further include instructions for: obtaining, based on a global background value, a binary image having a plurality of pixel values.

In some embodiments, the one or more programs further include instructions for: grouping, based on the plurality of pixel values, pixels of the binary image into the detected set of sequencing colonies.

In some embodiments, the one or more programs further include instructions for: determining a center pixel for each of the detected set of sequencing colonies.

In some embodiments, the one or more programs further include instructions for determining an initial location for each of the detected set of sequencing colonies.

In some embodiments, the initial location is a sub-pixel location.

In some embodiments, the determination comprises a center of mass estimation.

In some embodiments, the one or more programs further include instructions for: executing in parallel, using the graphics processor, a plurality of processes, each process corresponding to determining a respective sub-pixel location of a respective sequencing colony of the detected set of sequencing colonies.

In some embodiments, the one or more programs further include instructions for: registering a center patch of the input image and a center patch of a reference image to obtain a horizontal shift and a vertical shift of the input image with respect to the reference image.

In some embodiments, the reference image is an image in which all captured sequencing colonies emit signals over a predefined threshold.

In some embodiments, the registering comprises: generating a first synthetic image corresponding to the center patch of the input image; generating a second synthetic image corresponding to the center patch of the reference image; and correlating the first synthetic image with the second synthetic image.

In some embodiments, each sequencing colony in the center patch of the input image is represented by the same Gaussian profile in the first synthetic image.

In some embodiments, each sequencing colony in the center patch of the reference image is represented by the same Gaussian profile in the second synthetic image.

In some embodiments, correlating the first synthetic image with the second synthetic image comprises performing a two-dimensional cross correlation using Fourier transform.

In some embodiments, the one or more programs further include instructions for: generating an affine transformation between the reference image and the input image.

In some embodiments, the one or more programs further include instructions for: iteratively refining one or more coefficients of the affine transformation.

In some embodiments, the one or more programs further include instructions for: in each iteration: applying the affine transformation to the reference image; pairing one or more sequencing colonies in the input image with one or more transformed sequencing colonies in the reference image; and randomly selecting a number of paired sequencing colonies to refine the one or more coefficients of the affine transformation.

In some embodiments, the one or more programs further include instructions for: dividing the input image into a plurality of sub-images; identifying, for each sub-image of the plurality of sub-images, a group of pixels in the respective sub-image based on pixel-specific amplitude information; extending, for each sub-image, the respective group of pixels; calculating, for each sub-image, a local background value based on the extended respective group of pixels; and generating a background map based on local background values of the plurality of sub-images.

In some embodiments, the one or more programs further include instructions for: applying a mean filter to the background map.

In some embodiments, the one or more programs further include instructions for: deriving a colony-specific background for each detected sequencing colony of the detected set of sequencing colonies by bi-linear interpolation of the background map.

In some embodiments, the one or more programs further include instructions for: deriving a global background value based on a median of all extended groups of pixels for the plurality of sub-images.

In some embodiments, the one or more current profile properties include a current full width at half maximum (“FWHM”) estimate, a pseudo-Voigt Lorentzian weight (tail) parameter, or parameters of an elliptic model.

In some embodiments, the one or more current profile properties are determined based on an FWHM map.

In some embodiments, the surface is part of a substrate.

In some embodiments, the one or more programs further include instructions for: capturing an arc-shaped or ring-shaped image of the surface.

In some embodiments, the one or more programs further include instructions for: dividing the captured image into a plurality of image tiles, wherein the input image is one image tile of the plurality of image tiles.

In some embodiments, the one or more programs further include instructions for: executing in parallel, using the graphics processor, a plurality of processes, each process corresponding to a respective image tile of the plurality of image tiles.

In some embodiments, the one or more programs further include instructions for: detecting a plurality of sequencing colonies in a reference image; generating a simulated image based on the plurality of detected sequencing colonies in the reference image; subtracting the simulated image from the reference image to obtain a residual image; and detecting one or more additional sequencing colonies based on the residual image.

A non-transitory computer-readable storage medium storing one or more programs for determining nucleic acid sequences of a plurality of sequencing colonies, the one or more programs comprising instructions, which when executed by one or more processors of one or more electronic devices, cause the electronic devices to: obtain an input image of a surface, wherein the plurality of sequencing colonies are attached to the surface; detect a set of sequencing colonies of the plurality of sequencing colonies in the input image; execute in parallel, using a graphics processor, a plurality of iterative processes to obtain signal amplitudes for the detected set of sequencing colonies, wherein each iterative process corresponds to a respective detected sequencing colony in the set, and wherein each iterative process comprises: (a) obtaining amplitude, location, and profile estimates of one or more neighboring sequencing colonies to the respective sequencing colony; (b) calculating, using the graphics processor, a crosstalk value for the respective sequencing colony based on the amplitude, location, and profile estimates of the one or more neighboring sequencing colonies; (c) subtracting, using the graphics processor, the crosstalk value and a colony-specific background to obtain a current amplitude estimate of the respective sequencing colony; (d) performing a next iteration of (a)-(c) for a predetermined number of times or until a condition is met; and determine, at least partially based on the signal amplitudes for the detected set of sequencing colonies, portions of nucleic acid sequences of the plurality of sequencing colonies.

In some embodiments, each iterative process further comprises: determining, using the graphics processor, a current location estimate of the respective sequencing colony.

In some embodiments, each iterative process further comprises: determining, using the graphics processor, one or more current profile properties of the respective sequencing colony.

In some embodiments, the predetermined number of times is between 5-7 times.

In some embodiments, the input image is a first input image corresponding to a first flow step, the obtained signal amplitudes correspond to the first flow step, and the method further comprises: obtaining a second input image corresponding to a second flow step; and obtaining signal amplitudes corresponding to the second flow step.

In some embodiments, the one or more programs further comprise instructions for: identifying, based on the signal amplitudes corresponding to the first flow step and the second flow step, the nucleic acid sequences of the plurality of sequencing colonies.

In some embodiments, the plurality of sequencing colonies are attached to a plurality of beads attached to the surface.

In some embodiments, the one or more programs further comprise instructions for: capturing the input image of the surface.

In some embodiments, the one or more programs further comprise instructions for: combining the plurality of sequencing colonies with nucleotides before capturing the input image, wherein at least a portion of the nucleotides are labeled.

In some embodiments, detecting the set of sequencing colonies comprises: applying one or more filters to the input image.

In some embodiments, the one or more filters comprise a Gaussian filter.

In some embodiments, the Gaussian filter is based on a known profile of a standard bead attached to the surface.

In some embodiments, the known profile includes a shape, a size, or a full-width at half-maximum value of the standard bead.

In some embodiments, the one or more filters comprise a low-pass filter and/or a high-pass filter.

In some embodiments, the one or more programs further comprise instructions for: obtaining, based on a global background value, a binary image having a plurality of pixel values.

In some embodiments, the one or more programs further comprise instructions for: grouping, based on the plurality of pixel values, pixels of the binary image into the detected set of sequencing colonies.

In some embodiments, the one or more programs further comprise instructions for: determining a center pixel for each of the detected set of sequencing colonies.

In some embodiments, the one or more programs further comprise instructions for determining an initial location for each of the detected set of sequencing colonies.

In some embodiments, the initial location is a sub-pixel location.

In some embodiments, the determination comprises a center of mass estimation.

In some embodiments, the one or more programs further comprise instructions for: executing in parallel, using the graphics processor, a plurality of processes, each process corresponding to determining a respective sub-pixel location of a respective sequencing colony of the detected set of sequencing colonies.

In some embodiments, the one or more programs further comprise instructions for: registering a center patch of the input image and a center patch of a reference image to obtain a horizontal shift and a vertical shift of the input image with respect to the reference image.

In some embodiments, the reference image is an image in which all captured sequencing colonies emit signals over a predefined threshold.

In some embodiments, the registering comprises: generating a first synthetic image corresponding to the center patch of the input image; generating a second synthetic image corresponding to the center patch of the reference image; and correlating the first synthetic image with the second synthetic image.

In some embodiments, each sequencing colony in the center patch of the input image is represented by the same Gaussian profile in the first synthetic image.

In some embodiments, each sequencing colony in the center patch of the reference image is represented by the same Gaussian profile in the second synthetic image.

In some embodiments, correlating the first synthetic image with the second synthetic image comprises performing a two-dimensional cross correlation using Fourier transform.

In some embodiments, the one or more programs further comprise instructions for: generating an affine transformation between the reference image and the input image.

In some embodiments, the one or more programs further comprise instructions for: iteratively refining one or more coefficients of the affine transformation.

In some embodiments, the one or more programs further comprise instructions for: in each iteration: applying the affine transformation to the reference image; pairing one or more sequencing colonies in the input image with one or more transformed sequencing colonies in the reference image; and randomly selecting a number of paired sequencing colonies to refine the one or more coefficients of the affine transformation.

In some embodiments, the one or more programs further comprise instructions for: dividing the input image into a plurality of sub-images; identifying, for each sub-image of the plurality of sub-images, a group of pixels in the respective sub-image based on pixel-specific amplitude information; extending, for each sub-image, the respective group of pixels; calculating, for each sub-image, a local background value based on the extended respective group of pixels; and generating a background map based on local background values of the plurality of sub-images.

In some embodiments, the one or more programs further comprise instructions for: applying a mean filter to the background map.

In some embodiments, the one or more programs further comprise instructions for: deriving a colony-specific background for each detected sequencing colony of the detected set of sequencing colonies by bi-linear interpolation of the background map.

In some embodiments, the one or more programs further comprise instructions for: deriving a global background value based on a median of all extended groups of pixels for the plurality of sub-images.

In some embodiments, the one or more current profile properties include a current full width at half maximum (“FWHM”) estimate, a pseudo-Voigt Lorentzian weight (tail) parameter, or parameters of an elliptic model.

In some embodiments, the one or more current profile properties are determined based on an FWHM map.

In some embodiments, the surface is part of a substrate.

In some embodiments, the one or more programs further comprise instructions for: capturing an arc-shaped or ring-shaped image of the surface.

In some embodiments, the one or more programs further comprise instructions for: dividing the captured image into a plurality of image tiles, wherein the input image is one image tile of the plurality of image tiles.

In some embodiments, the one or more programs further comprise instructions for: executing in parallel, using the graphics processor, a plurality of processes, each process corresponding to a respective image tile of the plurality of image tiles.

In some embodiments, the one or more programs further comprise instructions for: detecting a plurality of sequencing colonies in a reference image; generating a simulated image based on the plurality of detected sequencing colonies in the reference image; subtracting the simulated image from the reference image to obtain a residual image; and detecting one or more additional sequencing colonies based on the residual image.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### DESCRIPTION OF THE FIGURES

The patent or application file contains at least one drawing executed in color. Copies of this patent or patent application publication with color drawings will be provided by the office upon request and payment of the necessary fee.

The invention will now be described, by way of example only, with reference to the accompanying drawings, in which:

FIG. 1 illustrates an exemplary flow sequencing method that can be used to generate sequencing data, in accordance with some embodiments.

FIG. 2A illustrates an exemplary summary of detected signals after a number of exemplary flow cycles are performed, in accordance with some embodiments.

FIG. 2B illustrates an exemplary process for determining a preliminary sequence, in accordance with some embodiments.

FIG. 3A illustrates a top view of an exemplary disc-shaped open substrate (also referred to as a wafer or a flow cell geometry) of a sequencing platform, in accordance with some embodiments.

FIG. 3B illustrates exemplary scanning path trajectories of an optical system, in accordance with some embodiments.

FIG. 4 illustrates an exemplary sub-image of an image tile of a portion of a substrate of a sequencing system, in accordance with some embodiments.

FIG. 5A illustrates an exemplary method for performing flow sequencing to determine a plurality of nucleic acid sequences of a plurality of sequencing colonies, in accordance with some embodiments.

FIG. 5B illustrates an exemplary set of outputs of the method, in accordance with some embodiments.

FIG. 6A illustrates an exemplary method for processing a reference image tile captured during flow sequencing, in accordance with some embodiments.

FIG. 6B illustrates an exemplary iterative process for determining one or more properties for a given sequencing colony, in accordance with some embodiments.

FIG. 7 illustrates an exemplary method for processing a flow image tile captured during flow sequencing, in accordance with some embodiments.

FIG. 8A illustrates exemplary background pixels identified within a sub-image of a reference image tile, in accordance with some embodiments.

FIG. 8B illustrates exemplary background pixels identified within a sub-image of a flow image tile, in accordance with some embodiments.

FIG. 9 illustrates various modes of an exemplary iterative process, in accordance with some embodiments.

FIG. 10A illustrates a histogram of true amplitudes of the sequencing colonies in an exemplary image, in accordance with some embodiments.

FIG. 10B illustrates an exemplary performance comparison, in accordance with some embodiments.

FIG. 10C illustrates an exemplary performance comparison, in accordance with some embodiments.

FIG. 11A illustrates an exemplary electronic device, in accordance with some embodiments.

FIG. 11B illustrates an example block diagram of information and processes that may be stored or used by device 1100, in accordance with some embodiments.

FIG. 11C illustrates an example block diagram of information that may be stored or used by device 1100, in accordance with some embodiments.

FIG. 11D illustrates an example block diagram of information that may be stored or used by device 1100, in accordance with some embodiments.

FIG. 12A illustrates how a larger sequencing colony profile and/or a larger amplitude variation among the sequencing colonies on a fairly dense surface (e.g., 90% load ratio) can negatively affect the performance of detection algorithms, in accordance with some embodiments.

FIG. 12B illustrates how residual image(s) can improve the performance of detection algorithms, in accordance with some embodiments.

FIG. 13A illustrates an exemplary process for processing an image tile captured during flow sequencing, in accordance with some embodiments.

FIG. 13B illustrates an exemplary reference image tile, in accordance with some embodiments.

FIG. 14A illustrates an exemplary histogram, in accordance with some embodiments.

FIG. 14B illustrates that the use of residual image(s) can improve the measurement of signal amplitudes, in accordance with some embodiments.

FIG. 15 illustrates an exemplary elliptic model for representing the profile of a sequencing colony, in accordance with some embodiments.

FIGS. 16A-16F illustrate that the use of an elliptic model can improve the measurement of signal amplitudes, in accordance with some embodiments.

FIG. 17 illustrates an example of additional beads detected by a second detection iteration as performed on a first flow image tile (e.g., a reference flow image tile), in accordance with some embodiments.

FIG. 18 illustrates an example of three types of beads identified in the registration stage of a typical sequencing flow, in accordance with some embodiments.

FIG. 19 illustrates an example of three types of beads identified in the registration stage for an all zero-mer flow, in accordance with some embodiments.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown but are to be accorded the scope consistent with the claims.

Disclosed herein are methods, electronic devices, systems, and non-transitory storage media for biological sample processing and/or analysis. In some embodiments, an exemplary system (e.g., one or more electronic devices) determines nucleic acid sequences of a plurality of sequencing colonies by first obtaining an input image of a surface that the plurality of sequencing colonies is attached to. The system detects one or more sequencing colonies of the plurality of sequencing colonies in the input image, and executes in parallel, using graphics processor(s), a plurality of iterative processes to obtain signal amplitudes, and in some embodiments other properties, for the plurality of sequencing colonies. Each iterative process corresponds to a respective detected sequencing colony of the one or more sequencing colonies in the input image, and each iterative process comprises: (a) obtaining amplitude, location, and profile estimates of one or more neighboring sequencing colonies to the respective sequencing colony from a previous iteration; (b) calculating, using the graphics processor, a crosstalk value for the respective sequencing colony based on the amplitude, location, and profile estimates of the one or more neighboring sequencing colonies; (c) subtracting, using the graphics processor, the crosstalk value and a background to obtain a current amplitude, and in some embodiments other properties, estimate of the respective sequencing colony; (d) performing a next iteration of (a)-(c) for a predetermined number of times or until a condition is met. The system can determine, at least partially based on the signal amplitudes for the plurality of sequencing colonies, nucleic acid sequences of the plurality of sequencing colonies.

Some embodiments of the present disclosure use an iterative process to refine the calculation of one or more properties of each sequencing colony. These properties may include signal amplitude, colony location, colony (or signal) profile, background, maximum gray-level, number of saturated pixels, local background, a measure of the goodness of fit of the colony (or signal) profile relative to a known profile, positional error, and/or a signal-to-noise ratio. In some embodiments, in each iteration, the system can determine a more refined estimate of the crosstalk for a sequencing colony, for example, using more refined estimated properties of neighboring sequencing colonies. The more refined estimate of the crosstalk allows the system to calculate a more refined estimate of the signal amplitude and other properties of the sequencing colony. In some embodiments, in each iteration, the system can additionally determine a more refined location of the sequencing colony and/or determine a more refined profile (e.g., full width at half maximum or FWHM value, profile tail behavior, profile distribution, etc.) of the sequencing colony. Iteratively refining multiple properties of the sequencing colonies lead to better understanding of the amount of signal crosstalk generated by neighboring sequencing colonies, thus allowing the system to provide more accurate signal amplitude estimates for each of the sequencing colonies.

Some embodiments of the present disclosure include generation of a background map and a global background value for an image by dividing the image into a plurality of sub-images and deriving background estimation for each sub-image. The techniques described herein are superior to conventional approaches, which typically involve simply masking or removing the detected objects and examining the remaining pixels. For an image that has a dense population of objects (e.g., sequencing colonies), the conventional approaches may remove most or all of the pixels. The remaining pixels may lead to detection errors, especially when the objects have relatively large profiles (e.g., high FWHM values) or are saturated, faint, or overlapping in the image.

Some embodiments of the present disclosure include generation of a profile map (e.g., a FWHM map and/or maps of profile properties, e.g., profile tail, profile asymmetry or ellipticity) for an image by dividing the image into a plurality of sub-images and deriving sub-image FWHM values. Generally, the profile of a sequencing colony near the center of an image tends to be smaller, while the profile of a sequencing colony near the edge of the image tends to be larger due to optical and mechanical imaging issues (e.g., auto-focus variations, optical aberrations such as coma, field curvature). The techniques described herein can calculate a FWHM value as an average of FWHM values of multiple sequencing colonies within a sub-image, thus correcting these issues.

Some embodiments of the present disclosure include a novel registration technique to align two images. Instead of aligning the images directly, the system can generate and align two synthetic images corresponding to the images. In each synthetic image, the objects (e.g., sequencing colonies) are represented using identical data representations, such that the varying amplitudes of the sequencing colonies do not affect the registration process (e.g., a sequencing colony having a stronger signal would not be weighted more heavily during the registration process). After correlating the synthetic images, the system may further refine the pairing using an iterative process. The refinement can be used to correct potential inaccuracies due to deformation and artifacts in the images (e.g., image deformation related to variations of scanning speed, angle, or location of the imager).

Some or all steps in all processes described herein can be performed using one or more GPUs using parallel processing. For example, each image can be processed simultaneously with another image; each image tile can be processed simultaneously with another image tile obtained at another, different time; each sequencing colony can be processed simultaneously with other sequencing colonies in the same image tile; each pixel can be processed simultaneously with other pixels in the same image tile.

Parallel processing significantly improves the throughput of the flow sequencing method. In one experiment, a flow sequencing method can involve hundreds of flow steps and each flow step can produce around one or more terabytes of image data. Embodiments of the present disclosure can process the image data at a high throughput (e.g., one or more gigabytes of image data per second). Further, the outputs are structured and stored in a memory-efficient manner. For example, for each flow, the system can store one or more bytes (e.g., 1 byte, 2 bytes, 4 bytes) of data for each sequencing colony's amplitude, one or more bytes (e.g., 1 byte, 2 bytes, 4 bytes) of data for each sequencing colony's location, and one or more bytes (e.g., 1 byte, 2 bytes, 4 bytes) of data for each sequencing colony's profile, in addition to a low-resolution background map and a low-resolution profile map as described herein.

Thus, embodiments of the present disclosure improve the functioning of computer systems and sequencing systems. Through novel data structures, processing logic, and use of GPUs, embodiments of the present disclosure provide improved memory usage, improved memory management, and improved processing to support the high-throughput requirement of the flow sequencing method to provide high-quality sequencing reads.

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X.”

A “flow order” refers to the order of separate nucleotide flows used to sequence a nucleic acid molecule using non-terminating nucleotides. The flow order may be divided into cycles of repeating units, and the flow order of the repeating units is termed a “flow-cycle order.” A “flow position” refers to the sequential position of a given separate nucleotide flow during the sequencing process.

The term “homopolymer length” refers to a number of sequential identical nucleotides of a particular base type in a nucleic acid sequence at a given flow step. The homopolymer length may be 0, 1, 2, 3 or any other 0 or positive integer value. A “homopolymer length likelihood” refers to a statistical parameter indicative of a likelihood or confidence that a given homopolymer length at a particular flow step is the correct homopolymer length.

The terms “individual,” “patient,” and “subject” can be used synonymously, and refers to an individual or entity from which a biological sample (e.g., a biological sample that is undergoing or will undergo processing or analysis) may be derived. A subject may be an animal (e.g., mammal or non-mammal) or plant. The subject may be a human, dog, cat, horse, pig, bird, non-human primate, simian, farm animal, companion animal, sport animal, or rodent. The subject may have or be suspected of having a disease or disorder, such as cancer (e.g., breast cancer, colorectal cancer, brain cancer, leukemia, lung cancer, skin cancer, liver cancer, pancreatic cancer, lymphoma, esophageal cancer, or cervical cancer) or an infectious disease. Alternatively, or in addition, a subject may be known to have previously had a disease or disorder. A subject may be undergoing treatment for a disease or disorder. A subject may be symptomatic or asymptomatic of a given disease or disorder. A subject may be healthy (e.g., not suspected of having disease or disorder). A subject may have one or more risk factors for a given disease. A subject may have a given weight, height, body mass index, or other physical characteristic. A subject may have a given ethnic or racial heritage, place of birth or residence, nationality, disease or remission state, family medical history, or other characteristic.

As used herein, the term “biological sample” generally refers to a sample obtained from a subject. The biological sample may be obtained directly or indirectly from the subject. A sample may be obtained from a subject via any suitable method, including, but not limited to, spitting, swabbing, blood draw, biopsy, obtaining excretions (e.g., urine, stool, sputum, vomit, or saliva), excision, scraping, and puncture. A sample may comprise a bodily fluid such as, but not limited to, blood (e.g., whole blood, red blood cells, leukocytes or white blood cells, platelets), plasma, serum, sweat, tears, saliva, sputum, urine, semen, mucus, synovial fluid, breast milk, colostrum, amniotic fluid, bile, bone marrow, interstitial or extracellular fluid, or cerebrospinal fluid. Alternatively, the sample may be obtained from any other source including but not limited to blood, sweat, hair follicle, buccal tissue, tears, menses, feces, or saliva of a subject. The biological sample may be a tissue sample, such as a tumor biopsy. The sample may be obtained from any of the tissues provided herein including, but not limited to, skin, heart, lung, kidney, breast, pancreas, liver, intestine, brain, prostate, esophagus, muscle, smooth muscle, bladder, gall bladder, colon, or thyroid. The biological sample may comprise one or more cells. A biological sample may comprise one or more nucleic acid molecules such as one or more deoxyribonucleic acid (DNA) and/or ribonucleic acid (RNA) molecules (e.g., included within cells or not included within cells). Nucleic acid molecules may be included within cells. Alternatively, or in addition, nucleic acid molecules may not be included within cells (e.g., cell-free nucleic acid molecules). The biological sample may be a cell-free sample.

The term “cell-free sample,” as used herein, generally refers to a sample that is substantially free of cells (e.g., less than 10% cells on a volume basis). A cell-free sample may be derived from any source (e.g., as described herein). For example, a cell-free sample may be derived from blood, sweat, urine, or saliva. For example, a cell-free sample may be derived from a tissue or bodily fluid. A cell-free sample may be derived from a plurality of tissues or bodily fluids. For example, a sample from a first tissue or fluid may be combined with a sample from a second tissue or fluid (e.g., while the samples are obtained or after the samples are obtained). In an example, a first fluid and a second fluid may be collected from a subject (e.g., at the same or different times) and the first and second fluids may be combined to provide a sample. A cell-free sample may comprise one or more nucleic acid molecules such as one or more DNA or RNA molecules.

The term “label,” as used herein, refers to a detectable moiety that is coupled to or may be coupled to another moiety, for example, a nucleotide or nucleotide analog. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease). In some embodiments, the label is a fluorophore.

The term “nucleotide,” as used herein, generally refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a modified or engineered nucleotide). A “non-terminating nucleotide” is a nucleic acid moiety that can be attached to a 3′ end of a polynucleotide using a polymerase or transcriptase, and that can have another non-terminating nucleic acid attached to it using a polymerase or transcriptase without the need to remove a protecting group or reversible terminator from the nucleotide. Naturally occurring nucleic acids are a type of non-terminating nucleic acid. Non-terminating nucleic acids may be labeled or unlabeled.

A “nucleotide flow” refers to a set of one or more non-terminating nucleotides (which may be labeled or a portion of which may be labeled).

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths, such as either deoxyribonucleotides or deoxyribonucleic acids (DNA) or ribonucleotides or ribonucleic acids (RNA), or analogs thereof.

Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid molecule can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), or more. A nucleic acid molecule (e.g., polynucleotide) can comprise a sequence of four natural nucleotide bases: adenine (A); cytosine (C); guanine (G); and thymine (T) (uracil (U) for thymine (T) when the polynucleotide is RNA). A nucleic acid molecule may include one or more nonstandard nucleotide(s), nucleotide analog(s) and/or modified nucleotide(s).

The term “sequencing,” as used herein, generally refers to a process for generating or identifying a sequence of a biological molecule, such as a nucleic molecule. Such sequence may be a nucleic acid sequence, which may include a sequence of nucleic acid bases. Sequencing may be single molecule sequencing or sequencing by synthesis, for example. Sequencing may be performed using template nucleic acid molecules immobilized on a support, such as a flow cell or one or more beads on a substrate as described herein.

When a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

Some of the analytical methods described herein include mapping sequences to a reference sequence, determining sequence information, and/or analyzing sequence information. It is well understood in the art that complementary sequences can be readily determined and/or analyzed, and that the description provided herein encompasses analytical methods performed in reference to a complementary sequence.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Generating Sequencing Data Using Flow Sequencing Methods

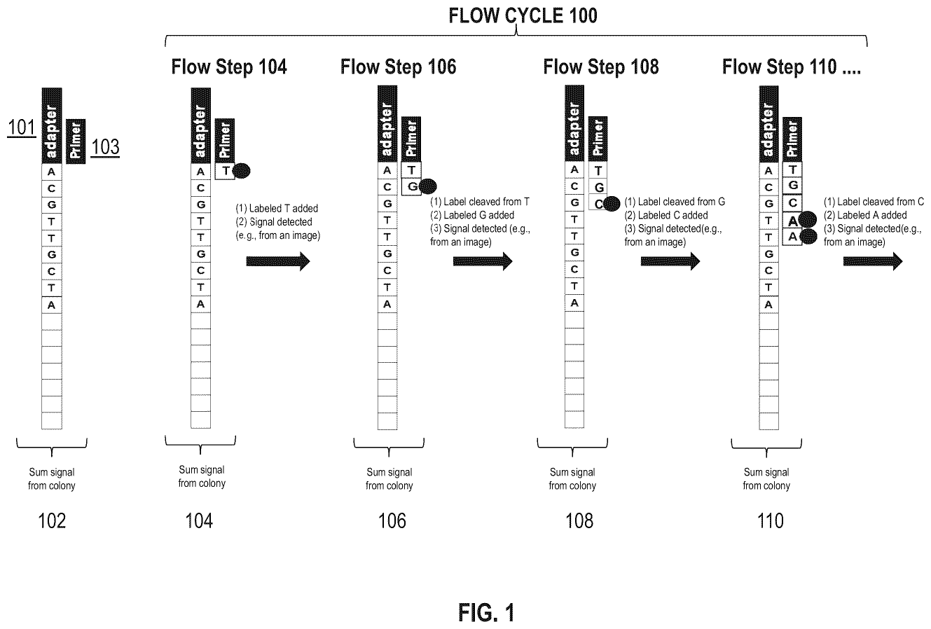

FIG. 1 illustrates an exemplary flow sequencing method that can be used to generate the sequencing data described herein. In some embodiments, polynucleotides may be bound to a surface (e.g., the surface of a bead attached to a substrate), as described in detail herein. The polynucleotides can include a nucleic acid sequence of interest (also referred to as a “template sequence”) and can further include a sequencing adapter sequence. The nucleic acid sequence of interest can be a nucleic acid molecule from or derived from a sample of a subject.

In the depicted example in FIG. 1, the nucleic acid sequence of interest includes an adapter sequence 101 followed by the nucleic acid sequence of interest (“ACGTTGCTA . . . ”).

The adapter sequence 101 can include a sequencing primer hybridization site. At step 102, a sequencing primer 103 is hybridized to the adapter sequence 101 of the polynucleotide at the sequencing primer hybridization site.

The sequencing primer is then extended in a series of flow cycles. In a flow cycle, the hybrid (i.e., the polynucleotide adapter hybridized to the sequencing primer) is combined with nucleotides (e.g., at least partially labeled nucleotides) and one or more signals indicating nucleotide incorporation into the sequencing primer may be detected. In the depicted example, the flow cycle 100 includes four flow steps 104, 106, 108, and 110. In a given flow step, a single type of nucleobase is combined with the hybrid according to the flow-cycle order T-G-C-A. As shown in FIG. 1, in flow step 104, labeled T nucleotides are combined with the hybrid; in flow step 106, labeled G nucleotides are combined with the hybrid; in flow step 108, labeled C nucleotides are combined with the hybrid; in flow step 110, labeled A nucleotides are combined with the hybrid.

At flow step 104, labeled T nucleotides are combined with the hybrid. Since the T base is complementary to the A base in the template polynucleotide, it is incorporated into the extending primer to form the hybrid as shown in 104. Further, a signal indicative of the incorporation of labeled T nucleotide into the sequencing primer can be detected. The signal may be detected, for example, by imaging the surface the polynucleotides are deposited on and analyzing the resulting image(s). In some embodiments, the sequencing platform may be washed with a wash buffer to remove unincorporated nucleotides prior to signal detection. In some embodiments, the detection of the signal is based on image processing techniques described herein.

At flow step 106, the label may be removed from the T nucleotide (e.g., by cleaving the label from the nucleotide). The sequencing method can then be continued with the next base in the flow order, G in the example illustrated in FIG. 1. At flow step 106, labeled G nucleotides are combined with the hybrid. Since the G base is complementary to the C base in the template polynucleotide, it is incorporated to form the hybrid in 106. Further, a signal indicating the incorporation of the labeled G nucleotide can be detected.

At flow step 108, the label may be removed from the G nucleotide (e.g., by cleaving the label from the nucleotide). The sequencing method can then be continued with the next base in the flow order, C. At flow step 108, labeled C nucleotides are combined with the hybrid. Since the C base is complementary to the G base in the template polynucleotide, it is incorporated into the extending primer to form the hybrid in 108. Further, a signal indicating the incorporation of the labeled C nucleotide into the sequencing primer can be detected.

At flow step 110, the label may be removed from the C nucleotide (e.g., by cleaving the label from the nucleotide). The sequencing method can then be continued with the next base in the flow order, A. At flow step 110, labeled A nucleotides are combined with the hybrid. Since the A base is complementary to the T base in the template polynucleotide, it is incorporated into the extending primer to form the hybrid in 110. Further, a signal indicating the incorporation of the labeled A nucleotide into the sequencing primer can be detected.

In flow step 110, because the template sequence includes two consecutive T bases, two A nucleotides are incorporated into the extending sequencing primer. Thus, the detected signal intensity indicating the incorporation of two A nucleotides may be greater than the signal intensity indicating the incorporation of one nucleotide.

While each flow step in the exemplary flow sequencing method in FIG. 1 results in incorporation of one or more nucleotides (and thus a detected signal indicating such incorporation), it should be appreciated that not all flow steps result in incorporation of nucleotides. In some flow steps, no nucleotide base may be incorporated (for example, in the absence of a complementary base in the template polynucleotide). For example, if C nucleotides are combined with a hybrid having a C base available for base pairing, no incorporation would occur and thus no signal indicative of an incorporation would be detected (e.g., because a G base would be required for base pairing with the C nucleotides). Further, as shown in flow step 110, two nucleotides or more than two nucleotides may be incorporated into the sequencing primer during an individual flow step for larger homopolymer lengths (e.g., greater than 1 nucleotide) in the nucleic acid sequence of interest. As another example, there may be one or more 0-mer (zero-mer) flows, where none or almost no sequencing colonies incorporate any nucleotides (e.g., sequencing colonies produce zero signal).

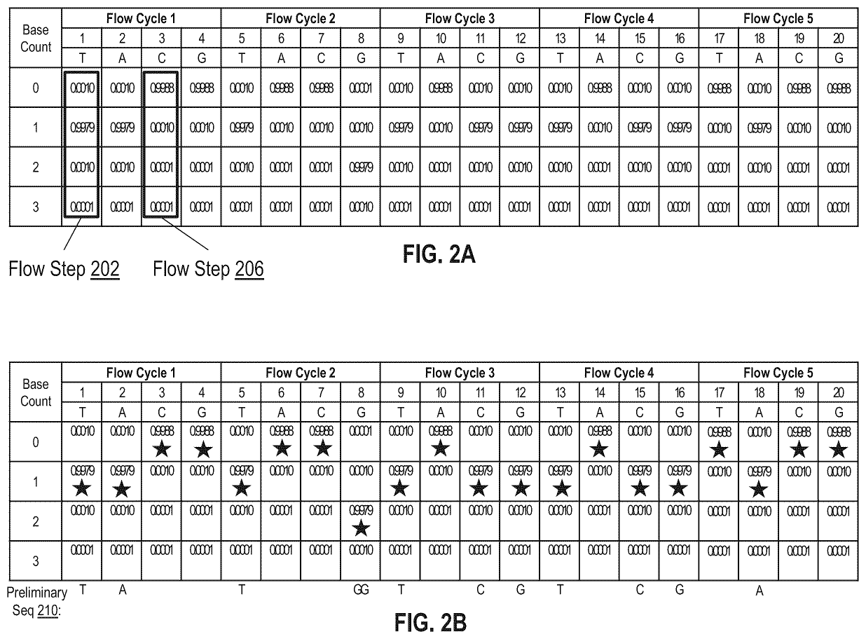

FIG. 2A illustrates an exemplary summary of detected signals after five exemplary flow cycles are performed, in accordance with some embodiments. Solely by way of example, a primer extended using a repeating flow-cycle order of T-A-C-G may result in a sequencing data flowgram set shown in FIG. 2A. Each column in FIG. 2A corresponds to a flow step and the values in each column collectively represent the detected signal intensity in the corresponding flow step, as described below.

In each flow step, the flow signal can be determined from an analog signal that is detected during the sequencing process, such as a fluorescent signal of the one or more bases incorporated into the sequencing primer during sequencing. Although an integer number of zero or more bases are incorporated at any given flow position, a given analog signal may not perfectly match with the analog signal. Therefore, in some embodiments, for a given flow step (e.g., flow step 202), the detected signal intensity can be expressed in probabilistic terms (e.g., with respect to homopolymer length). Specifically, the detected signal intensity can be expressed in four likelihood values corresponding to 0 base, 1 base, 2 bases, and 3 bases, respectively.

In the depicted example, for flow step 202, the detected signal intensity is expressed by a first likelihood value of 0.001 for 0 base, a second likelihood value of 0.9979 for 1 base, a third likelihood value of 0.001 for 3 bases, and a fourth likelihood value of 0.0001 for 4 bases. This can be interpreted to indicate that there is a high statistical likelihood that one nucleotide base has been incorporated. In the depicted example, the incorporation is a T since the flow step introduced labeled T nucleotides, which means there is an A in the template.

On the other hand, in flow step 206, the detected signal intensity is expressed by a first likelihood value of 0.9988 for 0 base, a second likelihood value of 0.001 for 1 base, a third likelihood value of 0.001 for 3 bases, and a fourth likelihood value of 0.0001 for 4 bases. This can be interpreted to indicate that there is a high likelihood that no nucleotide base has been incorporated. In the depicted example, no C has been incorporated.

Accordingly, the flowgram set in FIG. 2A is formatted as a sparse matrix, with a flow signal represented by a plurality of likelihood values indicating a plurality of likelihoods for a plurality of base homopolymer length counts (e.g., 0 base count, 1 base count, 2 base counts, and 3 base counts) at each flow position.

The homopolymer length likelihood may vary, for example, based on the noise or other artifacts present during detection of the analog signal during sequencing. In some embodiments, if the homopolymer length likelihood statistical parameter or likelihood is below a predetermined threshold, the parameter may be set to a predetermined non-zero value that is substantially zero (i.e., some very small value or negligible value) to aid the downstream statistical analysis further discussed herein, wherein a true zero value may give rise to a computational error or insufficiently differentiate between levels of unlikelihood, e.g., very unlikely (0.0001) and inconceivable (0).

With reference to FIG. 2B, a preliminary sequence can be determined based on the flowgram in FIG. 2A. For example, the most likely sequence can be determined by selecting the base count with the highest likelihood at each flow position, as shown by the stars in FIG. 2B. Thus, the preliminary sequence 210 can be determined as: TATGGTCGTCGA (SEQ ID NO: 1).

From the preliminary sequence (e.g., preliminary sequence 210), the reverse complement (i.e., the template strand or the nucleic acid sequence of interest) can be readily determined. Further, the likelihood of this sequencing data set, given the TATGGTCGTCGA (SEQ ID NO: 1) sequence (or the reverse complement), can be determined as the product of the selected likelihood (e.g., the most likely homopolymer length) at each flow position.

Accordingly, primer extension using flow sequencing allows for long-range sequencing on the order of hundreds or even thousands of bases in length. The number of flow steps or cycles can be increased or decreased to obtain the desired sequencing length. Extension of the primer can include one or more flow steps for stepwise extension of the primer using nucleotides having one or more different base types. In some embodiments, extension of the primer includes between 1 and about 1000 flow steps, such as between 1 and about 10 flow steps, between about 10 and about 20 flow steps, between about 20 and about 50 flow steps, between about 50 and about 100 flow steps, between about 100 and about 250 flow steps, between about 250 and about 500 flow steps, or between about 500 and about 1000 flow steps. The flow steps may be segmented into identical or different flow cycles. The number of bases incorporated into the primer depends on the sequence of the sequenced region (e.g., the template), and the flow order used to extend the primer. In some embodiments, the sequenced region is about 1 base to about 4000 bases in length, such as about 1 base to about 10 bases in length, about 10 bases to about 20 bases in length, about 20 bases to about 50 bases in length, about 50 bases to about 100 bases in length, about 100 bases to about 250 bases in length, about 250 bases to about 500 bases in length, about 500 bases to about 1000 bases in length, about 1000 bases to about 2000 bases in length, or about 2000 bases to about 4000 bases in length.

The output sequencing data set is uniquely structured to provide a computationally efficient analysis. The sequencing data set for the nucleic acid molecule colonies can include flow signals at flow positions that each corresponds to a flow of a particular nucleotide. Using this uniquely structured data set, the nucleic acid molecule (or molecules) can be analyzed in “flowspace” rather than “basespace” (also referred to as “nucleotide space” or “sequence space”). The flowspace data depend on additional information related to the flow-cycle order, which is not carried by basespace data. See, e.g., International published application WO 2020/227137 A1, which is incorporated herein by reference in its entirety.

Sequencing data can be generated using a flow sequencing method that includes extending a primer bound to a template nucleic acid molecule according to a pre-determined flow cycle or flow order where, in any given flow position, a type of nucleotide base is accessible to the extending primer. More commonly, a single type of nucleotide base is used in any given sequencing flow, although in some variations, two or three different types of nucleotide bases may be used, which allows for a faster primer extension but may provide less sequencing data about the sequence region. In some embodiments, at least some of the nucleotides of the particular type include a label, which upon incorporation of the labeled nucleotides into the extending primer renders a detectable signal. The resulting sequence by which such nucleotides are incorporated into the extended primer can be the reverse complement of the sequence of the template nucleic acid molecule, as described above with reference to FIG. 2B. For example, sequencing data may be generated using a flow sequencing method that includes extending a primer using labeled nucleotides and detecting the presence or absence of a labeled nucleotide incorporated into the extending primer. Flow sequencing methods may also be referred to as “natural sequencing-by-synthesis,” or “non-terminated sequencing-by-synthesis” methods. Exemplary methods are described in U.S. Pat. No. 8,772,473, International patent application WO 2021/007495 A1, International patent application WO 2020/227143 A1, and International patent application WO 2020/227137 A1, which are each incorporated herein by reference in their entirety. While the description herein is provided in reference to flow sequencing methods, it is understood that other sequencing methods may be used to sequence all or a portion of the sequenced region.

Flow sequencing includes the use of nucleotides to extend the primer hybridized to the nucleic acid molecule. Nucleotides of a given base type (e.g., A, C, G, T, U, etc.) can be mixed with hybridized templates to extend the primer if a complementary base is present in the template strand. The nucleotides may be, for example, non-terminating nucleotides. When the nucleotides are non-terminating, more than one consecutive base can be incorporated into the extending primer strand if more than one consecutive complementary base is present in the template strand. The non-terminating nucleotides contrast with nucleotides having 3′ reversible terminators, wherein a blocking group is generally removed before a successive nucleotide is attached. If no complementary base is present in the template strand, primer extension ceases until a nucleotide that is complementary to the next base in the template strand is introduced. At least a portion of the nucleotides can be labeled so that incorporation can be detected. In some embodiments, only a single nucleotide type is introduced at a time (i.e., discretely added), although two or three different types of nucleotides may be simultaneously introduced in some embodiments. This methodology can be contrasted with sequencing methods that use a reversible terminator, wherein primer extension is stopped after extension of every single base before the terminator is reversed to allow incorporation of the next succeeding base.

The nucleotides can be introduced at a determined order during the course of primer extension, which may be further divided into cycles. Nucleotides are added stepwise, which allows incorporation of the added nucleotide to the end of the sequencing primer of a complementary base in the template sequence. The cycles may have the same order of nucleotides and the same number of different base types or a different order of nucleotides and/or a different number of different base types. Solely by way of example, the order of a first cycle may be A-T-G-C and the order of a second cycle may be A-T-C-G. Alternative orders may be readily contemplated by one skilled in the art. Between the introductions of different nucleotides, unincorporated nucleotides may be removed, for example by washing the sequencing platform with a wash fluid.

A polymerase can be used to extend a sequencing primer by incorporating one or more nucleotides at the end of the primer in a template-dependent manner. In some embodiments, the polymerase is a DNA polymerase. The polymerase may be a naturally occurring polymerase or a synthetic (e.g., mutant) polymerase. The polymerase can be added at an initial step of primer extension, although supplemental polymerase may optionally be added during sequencing, for example with the stepwise addition of nucleotides or after a number of flow cycles. Exemplary polymerases include a DNA polymerase, an RNA polymerase, a thermostable polymerase, a wild-type polymerase, a modified polymerase, Bst DNA polymerase, Bst 2.0 DNA polymerase, Bst 3.0 DNA polymerase, Bsu DNA polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, and SeqAmp DNA polymerase.

The introduced nucleotides can include labeled nucleotides when determining the sequence of the template sequence, and the presence or absence of an incorporated labeled nucleic acid can be detected to determine a sequence. The label may be, for example, an optically active label (e.g., a fluorescent label) or a radioactive label, and a signal emitted by or altered by the label can be detected using a detector. The presence or absence of a labeled nucleotide incorporated into a primer hybridized to a template nucleic acid molecule can be detected, which allows for the determination of the sequence (for example, by generating a flowgram). In some embodiments, the labeled nucleotides are labeled with a fluorescent, luminescent, or other light-emitting moiety. In some embodiments, the label is attached to the nucleotide via a linker. In some embodiments, the linker is cleavable, e.g., through a photochemical or chemical cleavage reaction. For example, the label may be cleaved after detection and before incorporation of the successive nucleotide(s). In some embodiments, the label (or linker) is attached to the nucleotide base, or to another site on the nucleotide that does not interfere with elongation of the nascent strand of DNA. In some embodiments, the linker comprises a disulfide or PEG-containing moiety.

In some embodiments, the nucleotides introduced include only unlabeled nucleotides, and in some embodiments the nucleotides include a mixture of labeled and unlabeled nucleotides. For example, in some embodiments, the portion of labeled nucleotides compared to total nucleotides is about 90% or less, about 80% or less, about 70% or less, about 60% or less, about 50% or less, about 40% or less, about 30% or less, about 20% or less, about 10% or less, about 5% or less, about 4% or less, about 3% or less, about 2.5% or less, about 2% or less, about 1.5% or less, about 1% or less, about 0.5% or less, about 0.25% or less, about 0.1% or less, about 0.05% or less, about 0.025% or less, or about 0.01% or less. In some embodiments, the portion of labeled nucleotides compared to total nucleotides is about 100%, about 95% or more, about 90% or more, about 80% or more, about 70% or more, about 60% or more, about 50% or more, about 40% or more, about 30% or more, about 20% or more, about 10% or more, about 5% or more, about 4% or more, about 3% or more, about 2.5% or more, about 2% or more, about 1.5% or more, about 1% or more, about 0.5% or more, about 0.25% or more, about 0.1% or more, about 0.05% or more, about 0.025% or more, or about 0.01% or more. In some embodiments, the portion of labeled nucleotides compared to total nucleotides is about 0.01% to about 100%, such as about 0.01% to about 0.025%, about 0.025% to about 0.05%, about 0.05% to about 0.1%, about 0.1% to about 0.25%, about 0.25% to about 0.5%, about 0.5% to about 1%, about 1% to about 1.5%, about 1.5% to about 2%, about 2% to about 2.5%, about 2.5% to about 3%, about 3% to about 4%, about 4% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100%, or about 90% to about 100%.

### Exemplary Sequencing Platform

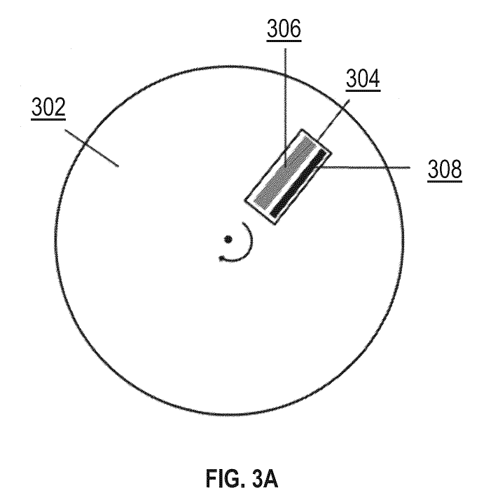

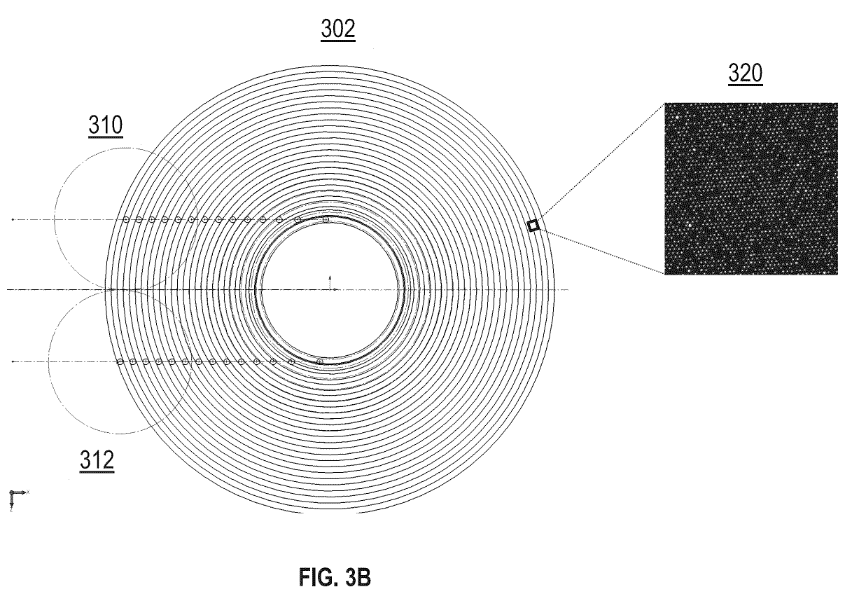

FIG. 3A illustrates a top view of an exemplary disc-shaped open substrate (also referred to as a wafer or flow cell geometry) of a sequencing platform. The sequencing platform can comprise one or more open substrates. The open substrates may be used to process any analyte, such as but not limited to, nucleic acid molecules, protein molecules, antibodies, antigens, cells, and/or organisms, as described herein. The open substrates or flow cell geometries may be used for any application or process, such as, but not limited to, sequencing by synthesis, sequencing by ligation, amplification, proteomics, single cell processing, barcoding, and sample preparation, as described herein.

In some embodiments, the sequencing platform described herein can be used to perform the flow sequencing method as described herein. First, a sequencing library can be prepared, and sequencing adapters (e.g., adapter sequence 101 in FIG. 1) can be ligated to the ends of the individual nucleic acids. The adapters serve as binding sites for primers (e.g., primer 103 in FIG. 1). In some embodiments, individual adapters can be engineered to contain unique molecule identifiers (UMIs), which can aid in downstream categorization or identification of the individual nucleic acid molecules and colonies.

The analyte to be processed (e.g., polynucleotides) may be coupled, attached, immobilized, or otherwise associated, directly or indirectly (e.g., via an intermediary object, such as a binder or linker) to an open substrate (e.g., substrate 300 in FIG. 3). For example, the polynucleotides may be coupled to a plurality of beads, which may be immobilized to the open substrate. In some embodiments, the beads are first attached to the substrate, then the polynucleotides are attached to the beads. In other embodiments, the polynucleotides are first attached to the beads and the beads are then attached to the substrate.

After polynucleotides are attached to the beads, amplification can be performed. In some embodiments, a colony is formed on each bead on the open substrate. In some embodiments, a colony comprises a plurality of nucleic acid molecules. In some embodiments, nucleic acid molecules in the plurality of nucleic acid molecules have sequence homology to a template sequence of the analyte. In some embodiments, each colony comprises amplified copies of a template sequence attached to the bead. While colony amplification may introduce errors that result in background signal noise, having many identical, amplified template nucleic acid molecules per bead/colony decreases the impact that any individual amplification error may have on the subsequent signal detection. In some embodiments, different beads on the substrate correspond to different template sequences.

In each flow step of the flow sequencing method (e.g., flow steps 104, 106, 108, 110 in FIG. 1), a combination of labeled and unlabeled nucleotides are introduced to the open substrate for sequencing reaction. For example, for each flow step, a solution of labeled and unlabeled nucleotides can be placed in the center of the substrate. The nucleotide solution can coat the substrate, and any excess solution can be removed. As described herein, the sequencing platform may be washed with a wash buffer to remove unincorporated nucleotides prior to signal detection.

In each flow step (e.g., flow steps 104, 106, 108, 110 in FIG. 1), the open substrate can be imaged after the nucleotides are introduced. The resulting image(s) can be analyzed to detect signals associated with the colonies on the substrate. In some embodiments, an optical imaging system is configured to scan the substrate while one of the optical imaging system and the substrate rotates, thus producing one or more images of ring, spiral, or arc shapes.

In the depicted example in FIG. 3A, the open substrate 302 rotates and a detector system 304 remains stationary during detection. Detector system 304 may comprise line-scan camera (e.g., TDI line-scan camera) 306 and illumination source 308. In some embodiments, the open substrate remains stationary, and a detector system rotates during detection. In some embodiments, other imaging schemes can be adopted to image the substrate or a portion thereof.

FIG. 3B illustrates exemplary optical path trajectories of an optical system (e.g., detector system 304 in FIG. 3A). In the depicted example, two imaging heads 310 and 312, each comprising an objective, may be positioned to image corresponding regions of the substrate 302. Accordingly, the optical system can produce one or more images via ring, spiral, or arc trajectories. In some embodiments, an image can be broken into a series of image tiles (e.g., image 320).

An exemplary substrate can comprise an array (such as a planar array) of individually addressable locations. In some instances, the array can be an array of wells. In some instances, the substrate can be textured and/or patterned. Each location, or a subset of such locations, may have immobilized thereto an analyte (e.g., a nucleic acid molecule, a protein molecule, a carbohydrate molecule, etc.). For example, an analyte may be immobilized to an individually addressable location via a support, such as a bead. A plurality of analytes immobilized to the substrate may be copies of a template analyte. For example, the plurality of analytes may have sequence homology. In other instances, the plurality of analytes immobilized to the substrate may be different. The plurality of analytes may be of the same type of analyte (e.g., a nucleic acid molecule) or may be a combination of different types of analytes (e.g., nucleic acid molecules, protein molecules, etc.). One or more surfaces of the substrate may be exposed to a surrounding open environment, and accessible from such surrounding open environment. For example, the array may be exposed and accessible from such surrounding open environment. In some cases, the surrounding open environment may be controlled and/or confined in a larger controlled environment.

The substrate may have the general form of a cylinder, a cylindrical shell or disk, a rectangular prism, or any other geometric form. The substrate may have a thickness (e.g., a minimum dimension) of at least 100 m, at least 200 m, at least 500 m, at least 1 mm, at least 2 mm, at least 5 mm, or at least 10 mm. The substrate may have a thickness that is within a range defined by any two of the preceding values. The substrate may have a first lateral dimension (such as a width for a substrate having the general form of a rectangular prism or a radius for a substrate having the general form of a cylinder) of at least 1 mm, at least 2 mm, at least 5 mm, at least 10 mm, at least 20 mm, at least 50 mm, at least 100 mm, at least 200 mm, at least 500 mm, or at least 1,000 mm. The substrate may have a first lateral dimension that is within a range defined by any two of the preceding values. The substrate may have a second lateral dimension (such as a length for a substrate having the general form of a rectangular prism) or at least 1 mm, at least 2 mm, at least 5 mm, at least 10 mm, at least 20 mm, at least 50 mm, at least 100 mm, at least 200 mm, at least 500 mm, or at least 1,000 mm. The substrate may have a second lateral dimension that is within a range defined by any two of the preceding values.

A surface of the substrate may be planar. A surface of the substrate may be uncovered and may be exposed to an atmosphere. Alternatively, or in addition, a surface of the substrate may be textured or patterned. For example, the substrate may comprise grooves, troughs, hills, and/or pillars. The substrate may define one or more cavities (e.g., micro-scale cavities or nano-scale cavities). The substrate may define one or more channels. The substrate may have regular textures and/or patterns across the surface of the substrate. For example, the substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. For example, the substrate may have any arbitrary structure above or below a reference level of the substrate. In some instances, a texture of the substrate may comprise structures having a maximum dimension of at most about 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar.

The substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. The one or more layers may have a thickness of at least 1 nanometer (nm), at least 2 nm, at least 5 nm, at least 10 nm, at least 20 nm, at least 50 nm, at least 100 nm, at least 200 nm, at least 500 nm, at least 1 micrometer (m), at least 2 m, at least 5 m, at least 10 m, at least 20 m, at least 50 m, at least 100 m, at least 200 m, at least 500 am, or at least 1 millimeter (mm). The one or more layers may have a thickness that is within a range defined by any two of the preceding values. A surface of the substrate may be modified to comprise any of the binders or linkers described herein. A surface of the substrate may be modified to comprise active chemical groups, such as amines, esters, hydroxyls, epoxides, and the like, or a combination thereof. In some instances, such binders, linkers, active chemical groups, and the like may be added as an additional layer or coating to the substrate.

The biological analyte may be any analyte that comes from a sample. For instance, the biological analyte may be a macromolecule, e.g., a nucleic acid molecule, a carbohydrate, a protein, a lipid, etc. The biological analyte may comprise multiple macromolecular groups, e.g., glycoproteins, proteoglycans, ribozymes, liposomes, etc. The biological analyte may be an antibody, antibody fragment, or engineered variant thereof, an antigen, a cell, a peptide, a polypeptide, etc. In some cases, the biological analyte comprises a nucleic acid molecule. The nucleic acid molecule may comprise at least about 10, 100, 1,000, 10,000, 100,000, 1,000,000, 10,000,000, 100,000,000, 1,000,000,000 or more nucleotides. Alternatively, or in addition, the nucleic acid molecule may comprise at most about 1,000,000,000, 100,000,000, 10,000,000, 1,000,000, 100,000, 10,000, 1,000, 100, 10 or fewer nucleotides. The nucleic acid molecule may have a number of nucleotides that is within a range defined by any two of the preceding values. In some cases, the nucleic acid molecule may also comprise a common sequence, to which an N-mer may bind. An N-mer may comprise 1, 2, 3, 4, 5, or 6 nucleotides and may bind the common sequence. In some cases, the nucleic acid molecules may be amplified to produce a colony of nucleic acid molecules attached to the substrate or attached to beads that may associate with or be immobilized to the substrate. In some instances, the nucleic acid molecules may be attached to beads and subjected to a nucleic acid reaction, e.g., amplification, to produce a clonal population of nucleic acid molecules attached to the beads.

Reagents may be dispensed to the substrate to multiple locations, and/or multiple reagents may be dispensed to the substrate to a single location, via different mechanisms. In some cases, dispensing (to multiple locations and/or of multiple reagents to a single location) may be achieved via relative motion of the substrate and the dispenser (e.g., a nozzle). For example, a reagent may be dispensed to the substrate at a first location, and thereafter travel to a second location different from the first location due to forces (e.g., centrifugal forces, centripetal forces, inertial forces, etc.) caused by motion of the substrate. In another example, a reagent may be dispensed to a reference location, and the substrate may be moved relative to the reference location such that the reagent is dispensed to multiple locations of the substrate. In some cases, dispensing (to multiple locations and/or of multiple reagents to a single location) may be achieved without relative motion between the substrate and the dispenser. For example, multiple dispensers may be used to dispense reagents to different locations, and/or multiple reagents to a single location, or a combination thereof (e.g., multiple reagents to multiple locations). In another example, an external force (e.g., involving a pressure differential), such as wind, may be applied to one or more surfaces of the substrate to direct reagents to different locations across the substrate. In another example, the method for dispensing reagents (e.g., to multiple locations and/or of multiple reagents to a single location) may comprise vibration. In such an example, reagents may be distributed or dispensed onto a single region or multiple regions of the substrate (or a surface of the substrate). The substrate (or a surface thereof) may then be subjected to vibration, which may spread the reagent to different locations across the substrate (or the surface). Alternatively, or in conjunction, the method may comprise using mechanical, electric, physical, or other means to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents. In some instances, where a volume of reagent is dispensed to the substrate at a first location, and thereafter travels to a second location different from the first location, the volume of reagent may travel in a path or paths, such that the travel path or paths are coated with the reagent. In some cases, such travel path or paths may encompass a desired surface area (e.g., entire surface area, partial surface area(s), etc.) of the substrate.

In some cases, the substrate may be rotatable about an axis. The analytes may be immobilized to the substrate during rotation. Reagents (e.g., nucleotides, antibodies, washing reagents, enzymes, etc.) may be dispensed onto the substrate prior to or during rotation (for instance, spun at a high rotational velocity) of the substrate to coat the array with the reagents and allow the analytes to interact with the reagents. For example, when the analytes are nucleic acid molecules and when the reagents comprise nucleotides, the nucleic acid molecules may incorporate or otherwise react with (e.g., transiently bind) one or more nucleotides. In another example, when the analytes are protein molecules and when the reagents comprise antibodies, the protein molecules may bind to or otherwise react with one or more antibodies. In another example, when the reagents comprise washing reagents, the substrate (and/or analytes on the substrate) may be washed of any unreacted (and/or unbound) reagents, agents, buffers, and/or other particles.

One or more signals (such as optical signals) may be detected from a detection area on the substrate prior to, during, or subsequent to, the dispensing of reagents to generate an output. For example, the output may be an intermediate or final result obtained from processing of the analyte. Signals may be detected in multiple instances. The dispensing, rotating (or other motion), and/or detecting operations, in any order (independently or simultaneously), may be repeated any number of times to process an analyte. In some instances, the substrate may be washed (e.g., via dispensing washing reagents) between consecutive dispensing of the reagents. One or more detection operations can be performed within a desired time frame. For example, the detection operation can be performed within about 1 minute, 50 seconds, 40 seconds, 30 seconds, 20 seconds, 10 seconds, or less than 10 seconds. In some instances, at least two detection operations can be performed within 1 minute, 50 seconds, 40 seconds, 30 seconds, 20 seconds, 10 seconds, or less than 10 seconds, etc. In some instances, at least three detection operations can be performed within 1 minute, 50 seconds, 40 seconds, 30 seconds, 20 seconds, 10 seconds, or less than 10 seconds.

Accordingly, in some embodiments, a solution is directed across the substrate and comes into contact with the biological analyte during rotation of the substrate. The solution may be directed in a radial direction (e.g., outwards) with respect to the substrate to coat the substrate and contact the biological analytes immobilized to the array. In some instances, the solution may comprise a plurality of probes. In some instances, the solution may be a washing solution. The biological analyte can be subjected to conditions sufficient to conduct a reaction between at least one probe of the plurality of probes and the biological analyte. The reaction may generate one or more signals from the at least one probe coupled to the biological analyte. The method can comprise detecting one or more signals, thereby analyzing the biological analyte.

In some instances, a solution can be dispensed to two or more different locations on the substrate and/or array. In some instances, multiple solutions can be dispensed to a single location on the substrate and/or array, such as using multiple dispensers. In some instances, the multiple solutions can be dispensed to multiple locations on the substrate and/or array. In some instances, a single solution can be dispensed to a single location. The substrate may be in relative motion with respect to one or more dispensers. The substrate may be stationary with respect to one or more dispensers. One or more dispensing operations can be performed within a desired time frame. For example, the dispensing operation can be performed within 1 minute, 50 seconds, 40 seconds, 30 seconds, 20 seconds, 10 seconds, or less than 10 seconds. In some instances, at least two dispensing operations can be performed within 1 minute, 50 seconds, 40 seconds, 30 seconds, 20 seconds, 10 seconds, or less than 10 seconds etc. In some instances, at least three dispensing operations can be performed within 1 minute, 50 seconds, 40 seconds, 30 seconds, 20 seconds, 10 seconds, or less than 10 seconds.

### Exemplary Signal Detection Techniques

Conventional techniques for detecting signal intensities of one or more objects captured in a given image typically involve identifying a peak amplitude associated with each object in the image. This simplistic approach can be inaccurate, inefficient, and computationally expensive, especially when processing images of biological samples such as images captured during flow sequencing.

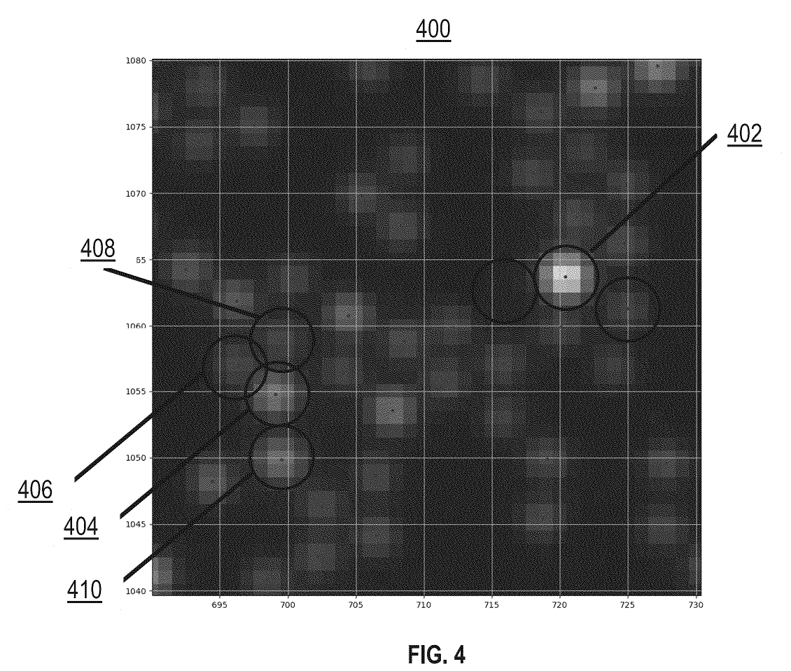

FIG. 4 shows an exemplary image tile 400 of a portion of a substrate of a sequencing system, in accordance with some embodiments. In some embodiments, the image tile 400 is the image 320 of FIG. 3B, which captures a portion of the substrate 302 as shown in FIG. 3B.

In some embodiments, the image tile 400 is captured during a flow step (e.g., any of flow steps 104, 106, 108, 110) after nucleotides are combined with sequencing colonies on the substrate. As described herein, the substrate can include a plurality of beads, and a sequencing colony can be formed on each bead of the plurality of beads. In some embodiments, a sequencing colony comprises a plurality of nucleic acid molecules. In some embodiments, nucleic acid molecules in the plurality of nucleic acid molecules have sequence homology to a template sequence. In some embodiments, each colony comprises amplified copies of the template sequence attached to the bead.

In the image tile 400, the brightness of each bead can be indicative of the signal intensity of the incorporated nucleotide(s) on the corresponding colony on the bead (e.g., of the number of incorporated nucleotides). Because each colony generally includes identical copies of the same polynucleotide, the colony-wise signal can be interpreted as the sum of all signals from the copies of the same polynucleotide in the colony. Thus, the intensity of the colony-wise signal can be indicative of how many labeled nucleotides have been incorporated, summed across the colony. In some rare instances, a colony will include one or more copies of one or more polynucleotides (i.e., a colony may be polyclonal to a varying extent). This may introduce some uncertainty into the interpretation of signal intensity with regards to the average number of labeled nucleotides that have been incorporated (i.e., this may be one factor as to why signal intensity values do not always correspond exactly to whole numbers of nucleotides incorporated).

In some embodiments, different colonies on a substrate can correspond to different template sequences. Thus, in a given flow step, the colonies on the substrate may have signals of varying intensities depending on whether the nucleotides applied in the flow step are incorporated in each of the colonies. Signal intensities in a given flow step further depend upon how many nucleotides applied in the flow step are incorporated into each colony with detectable brightness. For example, with reference to FIG. 4, the sequencing colony attached to bead 402 has a more intense signal than the sequencing colony attached to bead 404, which has a more intense signal than the sequencing colony attached to bead 408. Generally, a higher signal intensity indicate that the given sequencing colony has incorporated more labeled nucleotides from the flow step.

The conventional approach of determining signal intensities by simply examining the signal amplitudes (e.g., pixel-wise signal amplitudes) in the image can be ineffective and inaccurate when processing an image such as the image tile 400. Because a bead may be close to and/or overlap (e.g., with regards to the profile of each bead) with one or more neighboring beads, the neighboring beads can generate crosstalk or interference. For example, when a target bead is associated with a relatively weak signal (e.g., bead 406) but is located close to a neighboring bead with a stronger signal (e.g., bead 404), the stronger signal originating from the neighboring bead may be detected at the location associated with the target bead and be attributed to the target bead. Thus, the apparent signal amplitude of the target bead, based on the original image alone, would be higher than the actual signal amplitude of the target bead.

In some instances, a first bead has one or more neighboring beads. In some instances, the first bead has 1, 2, 3, 4, 5, or 6 neighboring beads. In some instances, a neighboring bead is within a set distance (e.g., a set number of microns, a set multiple of bead diameter, a set multiple of pitch size, etc.) of the first bead. In some instances, each of the one or more neighboring beads are within the set distance from the first bead. That is, the neighboring beads are each the set distance or less from the first bead. In some instances, a distance between a first bead and a second bead is defined as the distance center-to-center of the first bead to the second bead.

Further, the conventional approach, which relies on generic computer processors, is computationally expensive when processing images generated during flow sequencing. During flow sequencing, a large volume of images is generated at a high rate. For example, an exemplary flow sequencing method (e.g., the method shown in FIG. 1) may involve a large number of flow cycles (e.g., hundreds, thousands, tens of thousands, hundreds of thousands, millions of flow cycles), with each flow cycle comprising multiple flow steps. During each flow step, multiple images may be generated to capture the regions of interest on the substrate. In the depicted example in FIG. 3B, multiple ring-shaped images are generated during each flow step to capture the substrate. In some embodiments, each ring image may be cut into multiple image tiles (e.g., image 320 in FIG. 3B), generating a large number of image tiles (e.g., thousands, tens of thousands, hundreds of thousands of image tiles) in each flow step. Further, each image tile can be a high-definition image (e.g., thousands of pixels by thousands of pixels, tens of thousands of pixels by tens of thousands of pixels, hundreds of thousands of pixels by hundreds of thousands of pixels). Solely by way of example, during an exemplary flow step, about 30 ring images can be generated to capture the substrate. Each ring image may be cut into image tiles to generate 15,000 tiles during the flow step, each image tile being around 8,000 pixels by 2,000 pixels. The ring image can be a single-color image (e.g., greyscale image) or a color image. These images need to be processed at a high rate (e.g., thousands, tens of thousands, hundreds of thousands of images per second). The conventional approach relying on generic processors would not be able to process the images at such a high rate to support timely and efficient performance of the flow sequencing method.

Furthermore, a linear or serial process to process the image tiles (e.g., image tiles in a given flow step) one by one (e.g., processing only one image tile at a time before moving on to the next image tile) leads to an inefficient use of computer processing power and computer memory, while requiring a long processing time. Further still, each image tile (e.g., image tile 400 in FIG. 4) captures a plurality of sequencing colonies. A linear or serial process to process the sequencing colonies one by one in an image tile (e.g., detecting a sequencing colony, determining its signal intensity, and then moving on to detecting the next sequencing colony in the image tile) leads to an inefficient use of computer processing power and computer memory, while requiring a long processing time. For these additional reasons, the conventional approach relying on generic processors and linear processes would not be able to process the images at a high rate to support timely and efficient performance of the flow sequencing method.

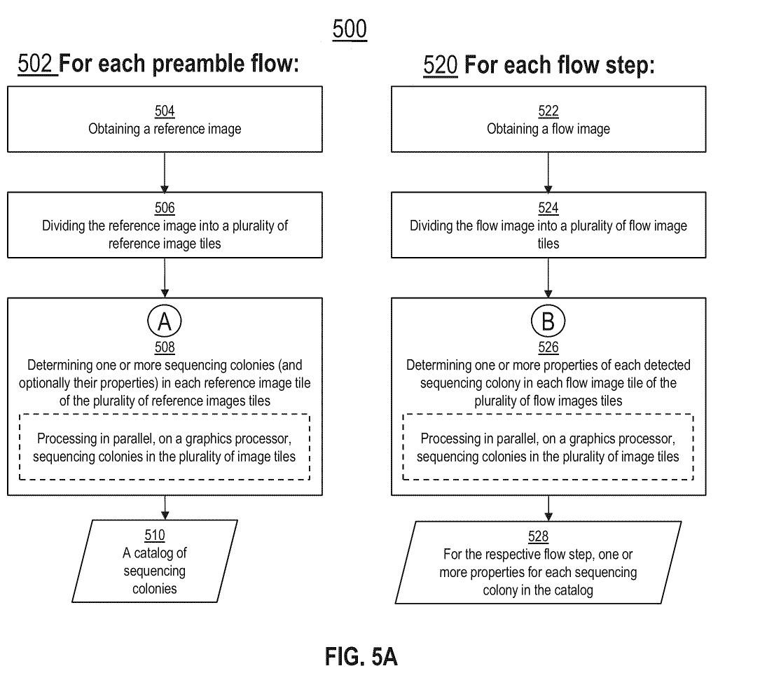

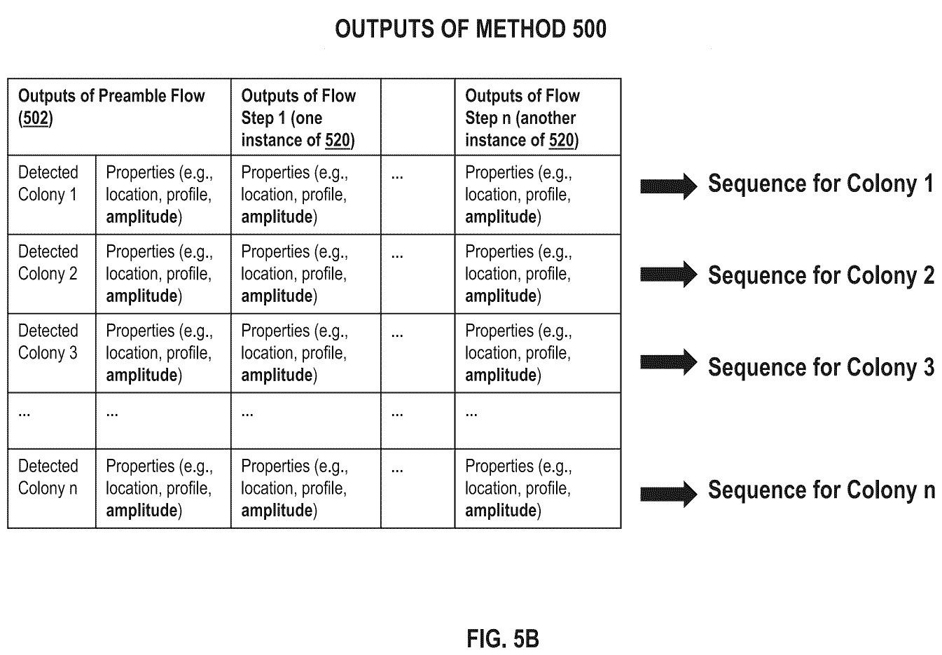

FIG. 5A illustrates an exemplary method for performing flow sequencing to determine a plurality of nucleic acid sequences of a plurality of sequencing colonies, in accordance with some embodiments. In some embodiments, method 500 is performed, for example, using one or more electronic devices implementing a software platform. In some examples, method 500 is performed using a client-server system, and the blocks of method 500 are divided up in any manner between the server and client device(s). In other examples, method 500 is performed using only a client device or only multiple client devices. In method 500, some blocks are, optionally, combined, the order of some blocks is, optionally, changed, and some blocks are, optionally, omitted. In some examples, additional steps may be performed in combination with the method 500. Accordingly, the operations as illustrated (and described in greater detail below) are exemplary by nature and, as such, should not be viewed as limiting.

The method 500 comprises a process 502 for processing reference image(s) from one or more preamble or reference flows and a process 520 for processing flow images from a given flow step. In some embodiments, the process 502 is performed once per preamble or reference flow to altogether determine a catalog of sequencing colonies 510 on a substrate or a portion thereof, and the process 520 is performed once per flow step to obtain one or more properties 528 for each sequencing colony in the catalog 510, as described below. In some embodiments, the process 502 can be performed for multiple times and the results can be integrated to obtain the catalog 510. In some embodiments, the process 502 can be optional and skipped. In some embodiments, the process 502 can be replaced with an alternative process for obtaining the catalog 510. For example, an exemplary alterative process can include aggregating detected sequencing colonies from several flows (e.g., 4 flows) to generate the catalog.

At block 504, an exemplary system (e.g., one or more electronic devices) obtains a reference image. The reference image captures a region of interest on the substrate to which the plurality of sequencing colonies is attached. In some embodiments, the reference image can be of a ring, spiral, or arc shape, as shown in FIG. 3B. At block 506, the system divides the reference image divided into a plurality of image tiles, as shown in FIG. 3B.

In some embodiments, in a reference image tile, all colonies captured in the image contain the same count of the same nucleotide, thus having a similar brightness level. For example, unlike the image tile 400 where the colonies have varying levels of brightness, all colonies in a reference image tile have a similar brightness level. In some embodiments, all colonies in the reference image tile are above a certain brightness threshold, within a certain range of brightness level, or a combination thereof. For example, all colonies in the reference image can provide a signal indicative of incorporation of one nucleotide base. In some embodiments, the brightness of all colonies in a reference image tile is similar, but not identical, due to many possible system variabilities (e.g., illumination pattern, different number of strands in each colony, variable colony size, etc.). In some embodiments, a reference image tile is used to identify all beads (e.g., sequencing colonies) for downstream analysis.

At block 508 (also referred to as “process A”), the system determines one or more sequencing colonies (and optionally their properties such as amplitude, location, profile, brightness, background, saturated pixels) in each image tile of the plurality of reference images tiles. In some embodiments, the reference image tiles are processed in parallel using one or more graphics processors (“GPUs”). In other words, a plurality of instances of process A corresponding to the plurality of reference image tiles can be performed simultaneously on one or more GPU units.

The preamble flow may result in multiple reference images (e.g., multiple ring images as shown in FIG. 3B). The reference images can be processed serially or in parallel using one or more GPU units. For example, multiple instances of process 502 can be performed simultaneously for all reference images in the preamble flow.

FIG. 5B illustrates an exemplary set of outputs of method 500, in accordance with some embodiments. With reference to FIG. 5B, the output of process 502 includes a catalog or list of sequencing colonies 1-n detected in all reference images from the preamble flow (i.e., all sequencing colonies on the substrate). The output of process 502 can further include or more properties associated with each detected sequencing colony. In some embodiments, the one or more properties include location data of the sequencing colony, profile data of the sequencing colony, amplitude, etc. Amplitude data can include a grey-level value that represents a 1-mer and can be compared against the amplitude in a later flow sequencing step to determine how many nucleotide bases have been incorporated into the sequencing primer. Location data can include, for example, a ring identifier, an image tile identifier, and location (e.g., pixel location of center, sub-pixel location of center) within the image tile. Profile data can indicate the size and/or shape of the sequencing colony and can include, for example, the FWHM values, moments, tails, etc. Additional properties of each sequencing colony may include for example its local/site background, peak brightness, saturated pixels count, etc.

During flow sequencing, a plurality of flow steps is performed as shown in FIG. 1. In each flow step, one or more flow images can be generated to capture the properties, for example signals, of the plurality of colonies on the substrate. At block 522, the system obtains a flow image. The flow image captures a region of interest on the substrate. In some embodiments, the flow image can be of a ring, spiral, or arc shape, as shown in FIG. 3B. At block 524, the system divides the flow image divided into a plurality of image tiles, as shown in FIG. 3B.

In the flow image tile, not all colonies captured by the image have a similar brightness level. For example, as shown in image tile 400 in FIG. 4, the colonies have varying levels of brightness indicative of incorporation of different numbers of nucleotide bases or no incorporation at all (e.g., dark or mostly dark colonies).

At block 526 (also referred to as “process B”), the system determines one or more properties of each detected sequencing colony in each image tile of the plurality of flow images tiles. In some embodiments, the flow image tiles are processed in parallel using one or more GPUs. In other words, a plurality of instances of process B corresponding to the plurality of flow image tiles can be performed simultaneously on a GPU or across multiple GPU units.

In method 500, each flow step may result in multiple flow images (e.g., multiple ring images as shown in FIG. 3B). The flow images can be processed serially or in parallel using a GPU or plurality of GPUs. For example, multiple instances of process 520 can be performed simultaneously for all flow images in the flow step. In some embodiments, images across multiple flow steps can be processed serially or in parallel using a GPU or plurality of GPUs.

With reference to FIG. 5B, in some instances, the output (e.g., colony properties 528 in FIG. 5A) of process 520 includes one or more properties associated with each sequencing colony in the catalog of sequencing colonies 510. In some embodiments, the one or more properties include location data of the sequencing colony, profile data of the sequencing colony, etc. Location data can include, for example, a ring identifier, an image tile identifier, and location within the image tile. Profile data can indicate the size and/or shape of the sequencing colony and can include, for example, the FWHM value. Addition properties of each sequencing colony may include for example its local/site background, peak brightness, saturated pixels count and others.

Further with reference to FIG. 5B, the outputs of method 500 can be used to determine a plurality of nucleic acid sequences of the sequencing colonies on the substrate (e.g., using the outputs of iterative process 520). For example, for each sequencing colony, the corresponding amplitudes of signals can be used to determine the nucleic acid sequence of the sequencing colony in accordance with the techniques described herein (e.g., with reference to FIGS. 1-2B). For example, for each sequencing colony, the corresponding amplitudes of signals can be translated into a flow diagram (e.g., the flow diagram in FIG. 2A), with each amplitude expressed in four likelihood values. The flow diagram can be then translated into a nucleic acid sequence as described herein. Nucleic acid sequencing may provide information that may be used to diagnose a certain condition in a subject and, in some cases, tailor a treatment plan. For example, nucleic acid sequencing may be used for cancer detection, treatment and recurrance detection. As another example, nucleic acid sequencing may be used for diagnosing heritary diseases. Sequencing can be used for molecular biology applications, including vector designs, gene therapy, vaccine design, industrial strain design and verification. Sequencing can be used to identify genomic DNA, RNA, or protein variants, mutations, and other inherited or environmental variations that may correspond to clinical conditions. Such information obtained from sequencing can further be used to direct therapy of such conditions.

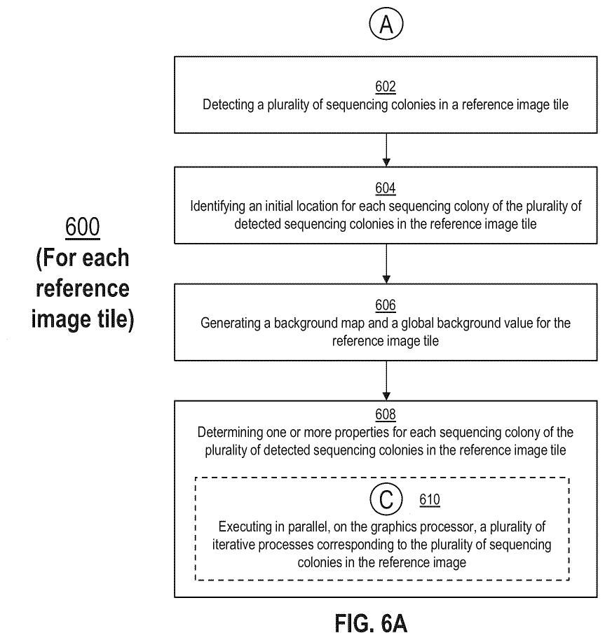

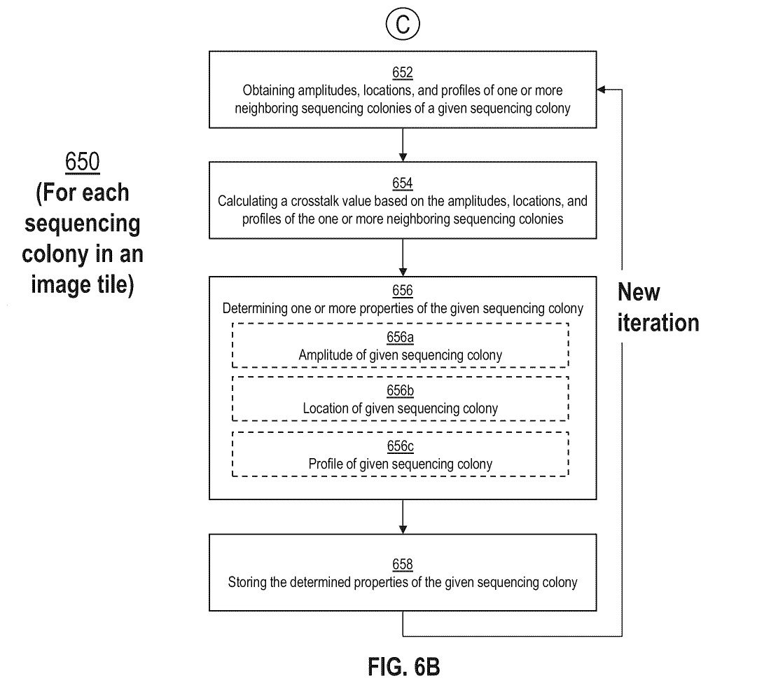

FIG. 6A illustrates an exemplary method 600 for processing a reference image tile captured during flow sequencing, in accordance with some embodiments. In some embodiments, the method 600 is block 508 or process “A” in FIG. 5A. In some embodiments, method 600 is performed, for example, using one or more electronic devices implementing a software platform. In some examples, method 600 is performed using a client-server system, and the blocks of method 600 are divided up in any manner between the server and client device(s). In other examples, method 600 is performed using only a client device or only multiple client devices. In method 600, some blocks are, optionally, combined, the order of some blocks is, optionally, changed, and some blocks are, optionally, omitted. In some examples, additional steps may be performed in combination with the method 600. Accordingly, the operations as illustrated (and described in greater detail below) are exemplary by nature and, as such, should not be viewed as limiting.

At block 602, an exemplary system (e.g., one or more electronic devices) detects a plurality of sequencing colonies in the reference image tile. In some embodiments, one or more pre-processing techniques can be first applied to the image tile, including identifying, removing, and/or adjusting undesirable regions and artifacts in the image tile.

In some embodiments, the system applies one or more filters to the image tile. The one or more filters can include a high-pass filter and/or a low-pass filter. The one or more filters can include a Gaussian filter. The Gaussian filter can be based on known or expected profile information of a standard bead attached to the substrate, such as a shape, a size, or a FWHM value of the standard bead. For example, the known or expected profile of a standard bead can be circular with a specific width, and the Gaussian filter can be set to optimize detection for the known or expected profile. Solely by way of example, a Gaussian filter can be 5 pixels by 5 pixels Gaussian filter with spatial sigma of 1 pixel (in which scenario, FWHM=2.35 pixels).

In some embodiments, the system can store the filter result after each filter is applied. For example, the system can first apply a high-pass filter to the image tile and store the first filter result (e.g., a first pixel map), and the system can then apply a Gaussian filter to the first filter result and store the second filter result (e.g., a second pixel map).

In some embodiments, the system can obtain a functional combination of the filter results (e.g., maximum, average). In some embodiments, after applying an adaptive threshold on the filter results, based on a derived global background value, the system can obtain a binary image having a plurality of pixel values. Solely by way of example, a pixel value of “0” can indicate no detection and a pixel value of “1” can indicate detection of the presence of a sequencing colony in the binary image. The global background value can be a proxy for the image noise level; thus, it can be used to define the detection threshold for the image tile. The detection threshold can be the square-root of the global background multiplied by a constant in some embodiments.

In some embodiments, the system groups, based on the plurality of pixel values, pixels of the binary image into the one or more detected sequencing colonies. For example, a cluster of neighboring pixel values of “1” can be grouped into a single detected sequencing colony.

In some embodiments, the system further determines a center pixel for each of the one or more detected sequencing colonies. In some embodiments, the system can store a pixel map in which the centers of the sequencing colonies are marked. For example, the pixel map can be a binary image in which only the centers of the sequencing colonies are valued at 1.

At block 604, the system identifies an initial location for each sequencing colony of the plurality of detected sequencing colonies in the reference image file. In some embodiments, the initial location is a pixel location. In some embodiments, the initial location is a sub-pixel location.

In some embodiments, the initial location is determined based on a center of mass estimation. For example, for each sequencing colony, the system obtains an image patch (e.g., a 3-pixel by 3-pixel patch) around the center pixel of the sequencing colony (e.g., as derived in block 602) and calculate the sub-pixel location based on the image patch using a center of mass estimation. As described below, the sub-pixel location can be refined further in block 608.

At block 606, the system generates a background map and a global background value for the reference image tile. To generate the background map for an image tile, the system can divide the image tile into a plurality of sub-images. Solely by way of example, an image tile that is 8,192 pixels by 2,048 pixels can be divided into a plurality of sub-images that are each 128 pixels by 128 pixels.

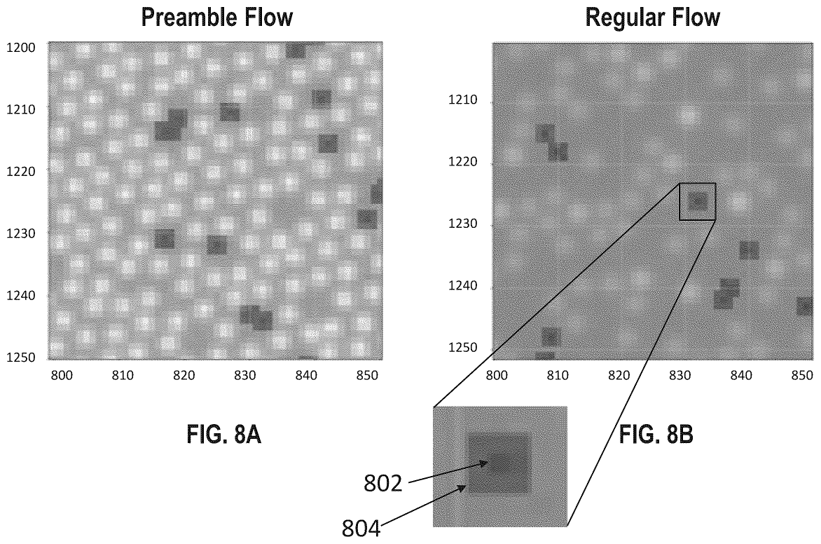

The system can then identify, for each sub-image of the plurality of sub-images, a group of pixels in the respective sub-image. In some embodiments, the system identifies, for each sub-image, a fraction (e.g., 0.25%) of the pixels having the lowest amplitudes (e.g., grey level values) and includes only those pixels in a group. The system can then extend, for each sub-image, the respective group of pixels. In some embodiments, for each group, the system adds, for each pixel in the group, its eight neighboring pixels to the group. FIG. 8A shows an exemplary sub-image of a reference image tile (e.g., from a preamble flow), and FIG. 8B shows an exemplary sub-image of a flow image tile (e.g., a regular flow). The pixels initially included in the group (i.e., the faintest pixels) are marked in dark grey (e.g., 802), and their neighboring eight pixels, which are included in the extended group, are marked in lighter grey (e.g., 804).

The system can then calculate, for each sub-image, a local background gray-level value based on the respective extended group of pixels. For example, the local background grey-level value can be calculated as the amplitude median of all pixels in the extended group. As another example, the local background grey-level value can be calculated as the amplitude median of all pixels in the extended group minus the original un-extended group of the faintest pixels.

The system can then generate a background map based on local background gray-level values of the plurality of sub-images. Thus, the background map is of a lower resolution than the image tile. Solely by way of example, if an image tile that is 8,192 pixels by 2,048 pixels is divided into a plurality of 128-by-128 sub-images, the background map would be 64 pixels by 16 pixels because each sub-image is represented as a single pixel in the background map. In some embodiments, a mean filter (e.g., a 3-by-3 mean filter) is then applied on the background map.

In some embodiments, the system derives a colony-specific background for each detected sequencing colony in the image tile by bi-linear interpolation (i.e., linear interpolation in 2 dimensions) of the background map. In some embodiments, this is done based on the exact location of the colony within the image tile determined in block 604 (e.g., the pixel or sub-pixel location).

In some embodiments, the system further derives a global background amplitude estimation based on a median of all extended groups of pixels for all sub-images in the image tile. The global background amplitude estimation can be used in block 602, as described above.

The techniques described in block 606 are superior to conventional approaches of obtaining a background map and a global background estimate. Conventional approaches can involve simply masking or removing the detected sequencing colonies and examining the remaining pixels. However, for an image tile that has a dense population of sequencing colonies, the conventional approaches may remove most or all of the pixels. In addition, some of the remaining pixels may still be illuminated (non-background pixels), especially when the beads have relatively large profiles (e.g., high FWHM values) or are saturated, faint, or overlapping in the image tile. These effects may result in non-determination, or wrong estimation, of the background level within each sub image, by conventional approaches.

At block 608, the system determines one or more properties for each sequencing colony of the plurality of detected colonies in the reference image tile. In some embodiments, at block 610, the system determines one or more properties (e.g., amplitude, location, profile, local background, saturated pixels) of each sequencing colony of the plurality of detected sequencing colonies in the reference image. In some embodiments, at block 610, the system executes a plurality of processes in parallel on the system's GPU. In other words, the plurality of processes can be executed simultaneously. The plurality of processes corresponds to the plurality of detected sequencing colonies, respectively, and each process is executed to obtain the one or more properties (e.g., amplitude, location, profile) of the respective sequencing colony. In some embodiments, each process is an iterative process comprising a plurality of iterations, as described with reference to FIG. 6B.

FIG. 6B illustrates an exemplary iterative process for determining one or more properties for a given sequencing colony, in accordance with some embodiments. In some embodiments, the process is one of the plurality of iterative processes in block 610 in FIG. 6A. In some embodiments, method 650 is performed, for example, using one or more electronic devices implementing a software platform. In some examples, method 650 is performed using a client-server system, and the blocks of method 650 are divided up in any manner between the server and client device(s). In other examples, method 650 is performed using only a client device or only multiple client devices. In method 650, some blocks are, optionally, combined, the order of some blocks is, optionally, changed, and some blocks are, optionally, omitted. In some examples, additional steps may be performed in combination with the method 650. Accordingly, the operations as illustrated (and described in greater detail below) are exemplary by nature and, as such, should not be viewed as limiting.

At block 652, an exemplary system (e.g., one or more electronic devices) obtains properties (e.g., amplitudes, locations, profiles, local background, saturated pixels) of one or more neighboring sequencing colonies of a given sequencing colony. Solely by way of example, in image tile 400 in FIG. 4, in the process corresponding to the sequencing colony on bead 404, the system can retrieve properties of neighboring colonies on beads including 406, 408, and 410. In some embodiments, the properties are retrieved from a memory unit.

At block 654, the system calculates a crosstalk value based on the amplitudes, locations, and profiles of the one or more neighboring sequencing colonies. The crosstalk value can comprise a patch or grid of pixel values, in which each pixel value represents the amplitude of crosstalk for the corresponding pixel. For example, for a central area of the given sequencing colony (e.g., a patch of 3 pixels by 3 pixels around the center pixel of the given sequencing colony), the system calculates the crosstalk in that central area by calculating an estimated patch of pixel values based on the properties of the neighboring beads (i.e., how strong and close the interfering sources are).

At block 656, the system determines one or more properties of the given sequencing colony. For example, the system can determine the amplitude of the given sequencing colony (e.g., block 656a), the location of the given sequencing colony (e.g., block 656b), or the profile of the given sequencing colony (e.g., block 656c). In some instances, the one or more properties may comprise an estimated amplitude, an estimated location, an estimated profile 656c (e.g., based on FWHM values), or an estimated local background value, of the given sequencing colony.

To determine an estimated amplitude 656a of the given sequencing colony, the system can first obtain a central area of the given sequencing colony in the image tile, and then subtract, from the central area, the crosstalk value, and the background map. For example, the system obtains a “clean” patch by taking a patch of the original image tile corresponding to the given sequencing colony and subtracting a patch of crosstalk values and a patch of the background map.

In some embodiments, the system identifies a patch of pixel values in the reference image tile that corresponds to the central area. The crosstalk value can be a patch of pixel values corresponding to the same pixels, and the background map can also be represented as a patch of pixel values corresponding to the same pixels. The background of a colony is a single value, interpolated by its location, from the background-map obtained in block 606 of FIG. 6A. For example, if a colony resides between two background sub-images, its background value can be calculated as the average of the two sub-images values.

The estimated amplitude can be derived by fitting the clean patch to a predefined sequencing colony model. The predefined sequencing colony model can be a Pseudo-Voigt model having a center amplitude of 1 grey-level and located at the same sub-pixel location. The system can then determine a multiplier of the predefined sequencing colony model that results in a close match to the clean patch. The multiplier can be assigned as the grey-level amplitude of the particular sequencing colony.

Since all sequencing colonies in the preamble flows represent 1-mer brightness these amplitude measurements can be used for normalizing the bead brightness by the base-calling process, in some embodiments. In some cases, the preamble may parallel the flow order (i.e., this may be how the uniform or substantially uniform 1-mer brightness may be produced as a result of preamble flows). For example, the preamble sequence that is included in sequencing colonies (e.g., as the first nucleotides prior to a sequence of interest) may be TGCA and the flow order may be T-G-C-A. In some instances, each preamble flow is used for normalization for future flows of a same nucleotide base. For example, a T preamble flow may be used by the base-calling process to normalize bead brightness during subsequent T flows.

To determine an estimated location 656b of the given sequencing colony, the system can first obtain a known profile of the sequencing colony. In some embodiments, the known profile is a predetermined constant FWHM value. In some embodiments, the known profile is obtained as a part of the iterative method 650 as described below with reference to 656c.

Given the known profile, the optimized sub-pixel location estimate is:

$$Odx = A * dx + B * Fb ( dx , dy ) + C * Fc ( dx , dy )$$

$$Ody = A * dy + B * Fb ( dy , dx ) + C * Fc ( dy , dx )$$

In the above equations, Odx is optimized dx, Ody is optimized dy, and dx is center-of-mass-delta x distance, dy is center-of-mass-delta y distance described above, all in pixel units, relative to the center pixel of the colony, and Fb and Fc are some functions of either dx, or dy, or both, that can be used to minimize the Odx and Ody errors Further, A, B, and C are fitted to minimize the Odx, Ody errors for the known profile. In other words, the system can optimize and a derive a more accurate Odx & Ody, based on the known profile (relative to the center-of-mass dx, dy that are generic and less accurate).

In each iteration, the updated location of the given sequencing colony is derived as:

$$newYX = w * optYX + ( 1 - w ) * prevYX$$

where w is some 0<w<1 weight that controls the weighted average of previous and newly measured colony location.

In the above equation, optYX is the measured optimized bead location of current iteration, prevYX is the previous iteration location, and newYX is the resulting current iteration location. The weight w can be a predefined constant between 0 and 1. In some embodiment, w equals 0.5.

To determine an estimated profile 656c of the given sequencing colony, the system can construct a FWHM map for the reference image tile. The reference image tile can be divided into a plurality of sub-images (e.g., sub-images of 512 pixels by 512 pixels). The FWHM map comprises one FWHM value for each sub-image, as described below.

For a given sub-image, for each sequencing colony in the sub-image, the crosstalk-subtracted 3×3 pixels of each sequencing colony are fitted to a 2D parabolic model using:

$$G L = A - B * {r}^{2} ,$$

where r² is the square pixel distance from center of sequencing colony. This calculation uses the optimized Odx and Ody, described above, as the center of the sequencing colony.

The FWHM value (in pixels) of the sequencing colony can be approximated as

$$\sqrt{\frac{2 A}{B}} .$$

For a given sub-image, the sub-image FWHM can be estimated as a weighted average of the FWHM values of the sequencing colonies in the sub-image, weighted by the amplitudes of the corresponding sequencing colonies. In some embodiments, only sequencing colonies whose amplitudes fall within a predefined range are used to calculate the weighted average. For example, only amplitudes of detected sequencing colonies within [minAmp, 0.8*(predefined saturation amplitude)] are used, thus excluding too faint or over-saturated sequencing colonies. In some embodiments, only sequencing colonies whose FWHM values fall within a predefined range are used to calculate the weighted average. For example, only colonies having FWHMs within the range [0.1*defaultFWHM, 1.9*defaultFWHM] are used, where defaultFWHM is a predefined constant, thus excluding FWHM values that deviate significantly from a known or expected default FWHM value. In some embodiments, a weighted average for a particular sub-image is included in the FWHM map only if the number of sequencing colonies used in the weighted average calculation exceeds a predefined threshold (e.g., 100). Otherwise, the average FWHM of all sub-images with measured FWHM (e.g., a neighboring sub-image) that meets the requirement is used for the particular sub-image in the FWHM map.

In each iteration, the updated FWHM value of each sub-image is derived as:

$$newFWHM = w * imgFWHM + ( 1 - w ) * prevFWHM$$

In the equation above, prevFWHM is the FWHM determined in the previous iteration. Further, imgFWHM is the FWHM measured in the current iteration, and the newFWHM is the resulting FWHM map of the current iteration The weight w is a predefined constant between 0 and 1 (e.g., 0.1, 0.2, 0.3, 0.4, 0.5, 0.8).

The use of a FWHM map provides a more accurate FWHM estimate for a given sequencing colony. Generally, the profile of a sequencing colony near the center of an image tends to be smaller, while the profile of a sequencing colony near the edge of an image tends to be larger due to imaging and optical issues (e.g., auto-focus variations, optical alignment, etc.). Thus, the FWHM value is calculated as a larger-scale average of FWHM values of multiple sequencing colonies within a sub-image, thus correcting these issues.

In some embodiments, the system uses a pseudo-Voigt profile model with two parameters: FWHM & Tail. The Pseudo-Voigt profile is defined as the weighted-average of a Gaussian & a Lorentzian of the same FWHM. For example:

$$Pseudo\_Voigt ( r , fwhm , tail ) = ( 1 - tail ) * Gauss ( r , fwhm ) + tail * Lorentz ( r , fwhm )$$

Where r is the 2D distance from center of the object spread function.

In some embodiments, at block 656c, the system represents profiles of sequencing colonies using an elliptic model to account for sequencing colonies that may not appear perfectly circular in images. The profile of a sequencing colony may not appear perfectly circular due to physical characteristics of the sequencing colony (e.g., size, shape), physical characteristics of the substrate (e.g., how close the sequencing colonies are to each other on the substrate), and/or distortions introduced by the optical system or during the imaging process. Further, the profile of a given sequencing colony may change (e.g., grow or deform) during a sequencing run. Thus, it would be advantageous to model the profiles of sequencing colonies in a precise manner.

In some embodiments, the system uses an elliptical pseudo-Voigt profile model with four parameters: a, b, c, and tail. The elliptic Pseudo-Voigt profile can be defined as the weighted-average of a Gaussian & a Lorentzian of the same (a, b, c). For example:

$$Pseudo\_Voigt = ( r , tail ) = ( 1 - tail ) * Gauss ( r ) + tail * Lorentz ( r ) ,$$

Where r=ax²+2bxy+cy², where x and y are the distances from the center of the profile.

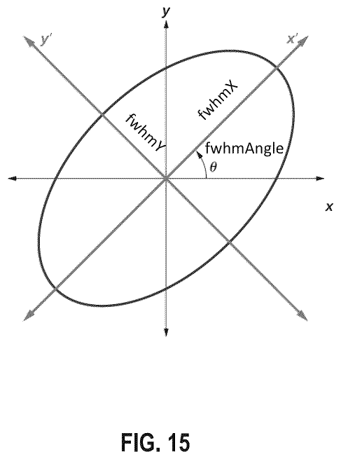

In some embodiments, the elliptical profile of a sequencing colony can be modeled either by the (a, b, c) representation or by three parameters: fwhmX, fwhmY and fwhmAngle (i.e., θ, the angle between ellipse-X and image-X directions), which are illustrated in FIG. 15. The two representations are interchangeable by a set of translation equations (e.g., a two-dimensional Gaussian function). It should be appreciated that the elliptic model can be used to model an elliptic shape (e.g., where fwhmX and fwhmY are different) and a circular shape (e.g., where fwhmX and fwhmY are identical). In some embodiments, fwhmX and fwhmY are pixel values. In some embodiments, fwhmAngle can be a degree value between −45 and 45.

As described above, to determine an estimated profile of the given sequencing colony, the system can construct an elliptic FWHM map for the image tile (e.g., a reference image tile or a flow image tile). The image tile can be further divided into a plurality of sub-images (e.g., sub-images of 512 pixels by 512 pixels as described elsewhere herein). The elliptic-FWHM map comprises the (fwhmX, fwhmY, fwhmAngle), or (a, b, c), values for each sub-image, as described below.

For a given sub-image, for each sequencing colony in the sub-image, the crosstalk-subtracted 3×3 pixels of each sequencing colony are fitted to a 2D parabolic model using:

$$GL = H * ( 1 - {ax}^{2} - 2 bxy - {cy}^{2} )$$

Where x and y are the pixel distances to the center of the sequencing colony. Accordingly, coefficients a, b, and c can be obtained for each sequencing colony in the sub-image. The coefficient α of a sub-image can be then estimated as the weighted average of the a values of all sequencing colonies in the sub-image weighted by the amplitudes of the corresponding sequencing colonies. Similarly, the coefficient b of a sub-image can be then estimated as the weighted average of the b values of all sequencing colonies in the sub-image weighted by the amplitudes of the corresponding sequencing colonies, and the coefficient c of a sub-image can be then estimated as the weighted average of the c values of all sequencing colonies in the sub-image weighted by the amplitudes of the corresponding sequencing colonies.

Sub-image fwhmX, fwhmY, and fwhmAngle are derived from the sub-image coefficients a, b, and c, using the translation equations.

In some embodiments, only sequencing colonies whose amplitudes fall within a predefined range are used to calculate the weighted average. For example, only amplitudes of detected sequencing colonies within [30, 0.8*(predefined saturation amplitude)] are used, thus excluding too faint or over-saturated sequencing colonies. In some embodiments, only sequencing colonies whose FWHM values fall within a predefined range are used to calculate the weighted average. For example, only sequencing colonies having a, b, c coefficients that translate to 0.1*defaultFWHM<FWHM<1.9*defaultFWHM are used, where defaultFWHM is a predefined constant, thus excluding FWHM values that deviate significantly from a known or expected default FWHM value. In some embodiments, defaultFWHM corresponds to 2.65, 3.6 for W, V, respectively. In some embodiments, a default FWHM can vary and to include a range that encompasses both the V and W values (e.g., about 0-5).

In some embodiments, the sub-image FWHM values (i.e., fwhmX, fwhmY, fwhmAngle) for a particular sub-image are included in the FWHM map only if the number of sequencing colonies used in calculating the values exceeds a predefined threshold (e.g., 100). Otherwise, a null is reported.

In each iteration, the updated FWHM coefficients of each sub-image can be derived as:

$$newABC = w * imgABC + ( 1 - w ) * prevABC ,$$

where imgABC corresponds to the coefficients a, b, and c derived above. The values a, b, c are bi-linear interpolated by their location on the image ellipABC (i.e., the (a, b, c) representation of the elliptic profile). Further, prevABC corresponds to the a, b, c coefficients from the previous iteration, and newABC corresponds to the a, b, c coefficients of the current iteration. The weight w is a predefined constant between 0 and 1 (e.g., 0.1, 0.2, 0.3, 0.4, 0.5, 0.8).

As described herein, the process C in FIG. 6B can be iterated for a plurality of times. In some embodiments, the process is iterated for a predefined number of times (e.g., 5, 6, 7 times). During the last iteration, amplitudes of all sequencing colonies can be estimated using the image mean coefficients a, b, and c. This prevents the FWHM estimation noise from increasing the output-signal noise.

The elliptic model provides a number of technical advantages. This approach does not rely on exact prior knowledge of the profiles of the sequencing colonies. Rather, the actual elliptic-FWHM pattern along an image is estimated and used for de-convolving the location and amplitude of the sequencing colonies. Further, changes of bead-profile elliptic FWHM in an image or across multiple images due to auto-focus variations, optical alignment, etc. are compensated for by adjusting the deconvolution-model elliptic profile.

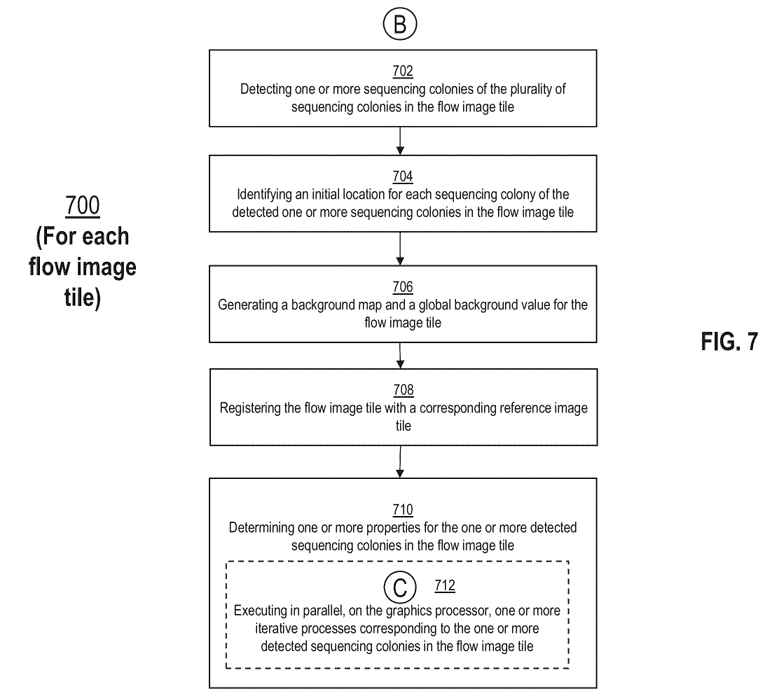

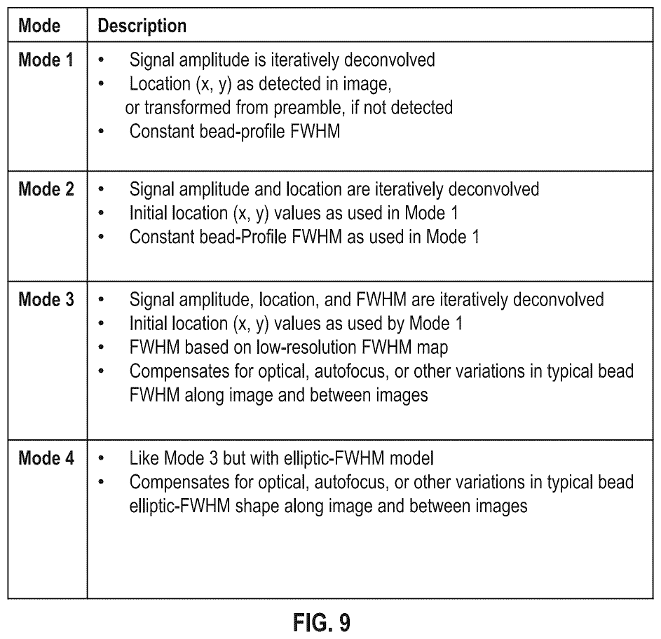

In some embodiments, the method 650 can be performed in four different modes, as shown in FIG. 9. Under Mode 1, only the amplitudes of sequencing colonies in the image tile are iteratively calculated. In other words, in each iteration, only 656a is calculated in block 656. For a reference image, the locations of the sequencing colonies can be generated in block 604 in FIG. 6A. For a flow image, the locations of the sequencing colonies can be assumed to be the same as those in the reference image, or they can be detected in block 704 in FIG. 7 as described below. Further, the profile FWHM values are assumed to be a predefined constant value.

Under Mode 2, the amplitudes and locations of sequencing colonies in the image tile are iteratively calculated. In other words, in each iteration, both 656a and 656b are calculated in block 656. The initial locations at the beginning of the iterations are assumed to be the same as the outputs of block 604 in FIG. 6A. Further, the profile FWHM values are assumed to be a predefined constant value.

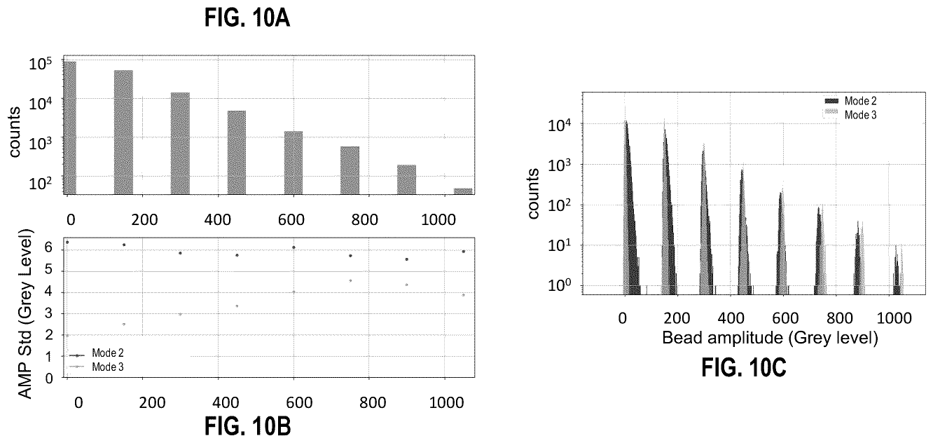

Under Mode 3, the amplitudes, the locations, and the profiles of the sequencing colonies in the image tile are iteratively calculated. In other words, in each iteration, 656a, 656b, and 656c are calculated in block 656. FIGS. 10A-10C provide exemplary performance comparisons between Mode 2 and Mode 3 based on a simulated image in which the properties of the sequencing colonies are known, according to some embodiments. FIG. 10A is a histogram of amplitudes of the sequencing colonies in the image, where the x axis represents the grey-level amplitudes. FIG. 10B shows amplitude standard deviations (in grey-level unit) corresponding to different amplitude levels. As shown, Mode 3 consistently produces a lower standard deviation across all amplitude levels. FIG. 10C shows a amplitude histogram. As shown, the amplitude spread associated with Mode 3 is narrower than Mode 2 across all amplitude levels, suggesting that Mode 3 produces more precise and consistent outputs.

Under Mode 4, the amplitudes, the locations, and the profiles of the sequencing colonies in the image tile are iteratively calculated in a manner similar to Mode 3. Further, an elliptic-FWHM model is used to account for bead shapes that are not perfectly circular, as described above with reference to block 656c in FIG. 6B. Mode 4 compensates for optical, autofocus, or other variations in typical bead elliptic-FWHM shape in a given image and between multiple images. It provides similar performance as Mode 3 with respect to circular bead profiles and provides improved performance with respect to non-circular bead profiles.

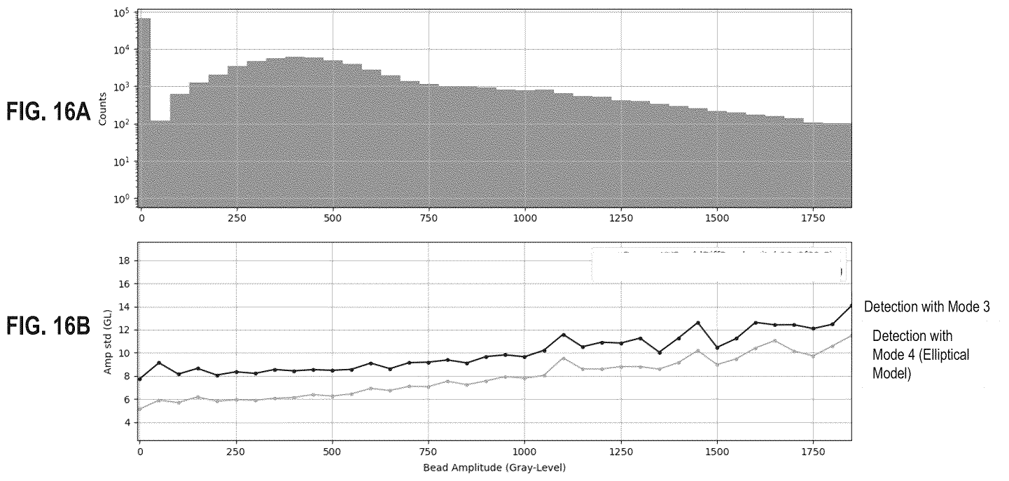

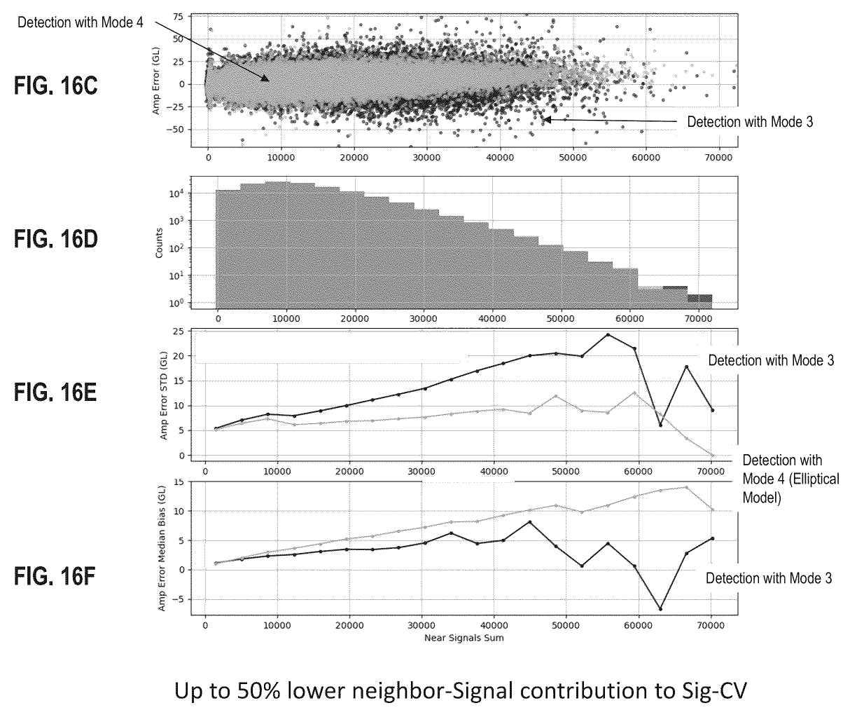

FIGS. 16A-16E provide exemplary performance comparisons between Mode 3 and Mode 4 based on a simulated image in which the properties of the sequencing colonies are known, according to some embodiments. In the simulated image used for the analyses, the average pitch was set to 1.8 μm with a 0.18 μm variance. The loading efficiency was set to 90% (e.g., 90% of the possible locations for a sequencing bead are occupied). The signal of each sequencing colony was set to a random homopolymer (e.g., indicative of a number of sequentially incorporated nucleotides into sequencing colonies) between 0 and 7, inclusive. The homopolymer values are converted to signal intensity (e.g., gray level) by multiplying by 400 (e.g., a homopolymer of 2 would have a signal intensity of 800 in this simulation). The FWHM for the sequencing colonies was set to x=1.27 μm and y=1.43 μm, with a θ=30°.

FIG. 16A illustrates an exemplary histogram in which the x-axis represents the various sequencing colony amplitudes, and the y-axis represents the number of detected sequencing colonies having a given amplitude. As shown, there is no difference between the number of sequencing colonies detected between Mode 3 and Mode 4, thus demonstrating that Mode 4 is not detrimental to the process of identifying sequencing colonies.

In FIG. 16B, the x-axis represents the various sequencing colony amplitudes, and the y-axis represents the amplitude standard deviation of the sequencing colonies at a given amplitude range. As shown, detection using the elliptic model (Mode 4) can lead to smaller amplitude deviations, suggesting more accurate amplitude measurements. Indeed, there is an approximately 25% reduction in amplitude deviations for Mode 4 as compared with Mode 3, across all amplitude levels (e.g., the signal measurement CV is reduced by approximately 25% by using Mode 4 over Mode 3).

FIGS. 16C-16F further illustrate the improved performance of Mode 4 in comparison with Mode 3, specifically with regards to the impact of neighboring sequencing colonies. FIG. 16C shows an exemplary amplitude error scatterplot. As shown, the amplitude error spread associated with neighboring sequencing colonies (e.g., ‘near signals sum’) with Mode 4 is narrower than that observed in Mode 3 across all signal levels of neighboring sequencing colonies, suggesting that Mode 4 produces more precise and consistent outputs. FIG. 16D illustrates an exemplary histogram in which the x-axis represents the various amplitudes of neighboring sequencing colonies, and the y-axis represents the number of detected sequencing colonies having neighboring sequencing colonies with a given amplitude. As seen in FIG. 16D, there is very little difference in the number of sequencing colonies detected by Mode 4 versus Mode 3 across all neighboring colony amplitudes (e.g., similar to the results observed in FIG. 16A). In FIG. 16E and FIG. 16F, the x-axis represents the various neighboring sequencing colony amplitudes (e.g., sums of all neighboring sequencing colony amplitudes for a given detected sequencing colony), and the y-axis represents the amplitude standard deviation (FIG. 16E) and median bias (FIG. 16F) of the sequencing colonies at a given neighboring colony amplitude. Using Mode 4 can provide up to approximately 50% reduction in sequencing colony amplitude standard deviation.

Turning back to FIG. 6B, at block 658, the system stores (e.g., to a memory unit) the determined properties of the given sequencing colony. A new iteration can start from block 652. The stored values can be retrieved from the memory unit in the next iteration for the given sequencing colony (e.g., as the previous iteration amplitude, the previous iteration location, the previous iteration profile), or can be retrieved from the memory unit in an iterative process corresponding to a neighboring sequencing colony (e.g., to calculate the crosstalk value to that neighboring sequencing colony in block 654).

The iterative method 650 can be terminated after a predefined number of iterations (e.g., 4, 5, 6, 7, 8, 10, 20, 100, etc.) are performed, or when a condition is met. In some embodiments, the condition is that the differences (e.g., the sum of squares of the differences) between the amplitudes determined in current and previous iterations are smaller than a predefined threshold. At the end of the method 650, the system stores the determined one or more properties of the given sequencing colony as a part of a catalog of sequencing colonies 510 (FIG. 5A). For example, the system can designate the given sequencing colony as “Detected Colony 1” and store its associated properties, as shown in FIG. 5B.

FIG. 7 illustrates an exemplary method 700 for processing a flow image tile captured during flow sequencing, in accordance with some embodiments. In some embodiments, the method 700 is block 526 or process “B” in FIG. 5A. In some embodiments, method 700 is performed, for example, using one or more electronic devices implementing a software platform. In some examples, method 700 is performed using a client-server system, and the blocks of method 700 are divided up in any manner between the server and client device(s). In other examples, method 700 is performed using only a client device or only multiple client devices. In method 700, some blocks are, optionally, combined, the order of some blocks is, optionally, changed, and some blocks are, optionally, omitted. In some examples, additional steps may be performed in combination with the method 700. Accordingly, the operations as illustrated (and described in greater detail below) are exemplary by nature and, as such, should not be viewed as limiting.

At block 702, an exemplary system (e.g., one or more electronic devices) detects one or more sequencing colonies in the flow image tile. The detection can be performed using techniques identical or similar to those described with reference to block 602 in FIG. 6A. It should be appreciated that, unlike a reference image tile in which all captured sequencing colonies emit signals of similar amplitudes, in a flow image tile, the sequencing colonies may emit signals of varying amplitudes, and some sequencing colonies may not emit any detectable signals at all and thus are not detected in block 702. In other words, in some embodiments, only a subset of the sequencing colonies captured in the flow image tile is detected in block 702.

At block 704, the system identifies an initial location for each sequencing colony of the detected one or more sequencing colonies in the flow image tile. In some embodiments, the initial location is a sub-pixel location. The identification can be performed using techniques identical or similar to those described with reference to block 604 in FIG. 6A.

At block 706, the system generates a background map and a global background value for the flow image tile. This can be performed using techniques identical or similar to those described with reference to block 606 in FIG. 6A.

At block 708, the system registers the flow image tile with a corresponding reference image tile that has been processed in process 502 (FIG. 5A). Although the flow image tile and the corresponding reference image tile are configured to capture the same portion of the substrate, the subject in the flow image tile may have shifted relative to the reference image tile due to, for example, mechanical deviations (e.g., movement of the imager and/or the sample). Thus, block 708 is performed to obtain a pairing between each sequencing colony in the flow image and the corresponding sequencing colony in the reference image.

In some embodiments, the system registers a center sub-image of the flow image tile and a center sub-image of a reference image tile to obtain a global horizontal shift and a global vertical shift of the flow image tile with respect to the reference image tile. As discussed below, instead of aligning the two center sub images directly, the system can generate and align two synthetic images corresponding to the two center sub images. In each synthetic image, the sequencing colonies are represented using identical data representations, such that the varying amplitudes of the sequencing colonies do not affect the registration process (e.g., a sequencing colony having a stronger signal would not be weighted heavier during the registration process).

For example, the system can first generate a first synthetic image corresponding to the center sub-image of the flow image tile. The center sub-image, for example, can be 1,000 pixels by 1,000 pixels at or around the center of the flow image. In the first synthetic image, each sequencing colony in the center sub-image is represented, e.g., by the same Gaussian profile. For example, the first synthetic image can be initialized such that each pixel value is 0. Then, the system can insert an identical standard Gaussian profile at the location of each detected sequencing colony in the flow image tile. The inserted standard Gaussian profiles can have the same properties, such as the same amplitude (e.g., 1), and the same standard deviation (e.g., 1).

The system can then generate a second synthetic image corresponding to the center sub-image of the reference image tile. The center sub-image, for example, can be of 1,000 pixels by 1,000 pixels at or around the center of the reference image. In the second synthetic image, each sequencing colony is represented by the same Gaussian profile. For example, the second synthetic image can be initialized such that each pixel value is 0. Then, the system can insert an identical standard Gaussian profile at the location of each detected sequencing colony in the reference image tile. The inserted standard Gaussian profiles can have the same properties, such as the same amplitude (e.g., 1), and the same standard deviation (e.g., 1).

The system can then correlate the first synthetic image with the second synthetic image. In some embodiments, the system identifies a horizontal shift gₓ (i.e., x) and a vertical shift g_{y} (i.e., y), in pixel units, which would produce the maximum overlap between the two synthetic images. In some embodiments, correlating the first synthetic image with the second synthetic image comprises performing a two-dimensional cross correlation using Fourier transform.

After correlating the first synthetic image with the second synthetic the system tries to pair each bead in the flow image to a reference bead, shifted by a distance (gₓ, g_{y}) (e.g., an affine transformation). Such pairing is defined as successful if the distance between the flow bead and the shifted reference bead is less than a predefined search radius (e.g., 1.5, 2.0, 2.5, or 3 pixels). Using the precise locations of the paired flow-reference beads, the system may refine the affine transformation. The refinement may be needed to correct potential inaccuracies due to deformation and artifacts in the images (e.g., image deformation related to scanning speed, location inaccuracies, or rotation of the imager).

In some embodiments, to refine the affine transformation, the system iteratively pairs the flow image colonies to the reference image colonies, shifted by previous iteration transformation coefficients, and uses the paired precise locations to further refine one or more coefficients of the affine transformation. In each iteration, the system applies the affine transformation to the reference image or reference bead locations. The system then pairs one or more detected sequencing colonies in the flow image tile with the corresponding transformed sequencing colonies in the reference tile and uses the paired precise locations to further refine one or more coefficients of the affine transformation. In some embodiments, pairing is based on a constant maximum distance between a colony location in the flow image to the transformed location of the reference image colony. For example, if the distance between the two colonies is smaller than a predefined threshold (e.g., number of pixels), the two sequencing colonies are paired. In some embodiments, during one or more initial iterations, mapping is limited to a center portion of the reference image tile and a center portion of the flow image tile (e.g., 1,000 pixels by 1,000 pixels). This enables support for larger deformation coefficients.

After the sequencing colonies in the flow image tile are paired with sequencing colonies in the reference image tile, the system randomly selects a number of paired sequencing colonies to refine the coefficients of the affine transformation. In some embodiments, the new registration and pairing is based on affine transformation:

$${Y}_{i} - {Y}_{ref} = {g}_{y} + {A}_{yy} * {Y}_{REF} + {A}_{yx} * {X}_{REF} ;$$

$$and$$

$${X}_{i} - {X}_{ref} = {g}_{x} + {A}_{xy} * {Y}_{REF} + Axx * {X}_{REF}$$

In the above equations, (g_{y}, gₓ, A_{yy}, A_{yx}, A_{xy}, Aₓₓ) are the constant transformation coefficients for the flow image to be refined. In some embodiments, coefficients measure the image deformation, in pixels, on image edges. In the initial iteration, the values of gₓ and g_{y} are the global horizontal shift and vertical shift derived from the correlation of synthetic images, and (A_{yy}, A_{yx}, A_{xy}, Aₓₓ) are all zeros.

Further, (Y_{ref}, X_{ref}) and (Yᵢ, Xᵢ) are colony locations in the reference image tile and the flow image tile, respectively. Further, (Y_{REF}, X_{REF}) are reference image colony locations normalized to a [−1,1] range.

In the next iteration, pairing and coefficient refinement based on randomly selected sequencing colonies are performed again. The iterations can be performed for a predefined number of times, or until a condition is met. In some embodiments, registration is an optional step and is not performed for all flow image tiles. For example, registration can be performed for only one image tile in a flow image, and the global shifts and coefficients can be applied to all other image tiles from the same ring flow image (e.g., because they share the same mechanical deviations).

At block 710, the system determines one or more properties for each sequencing colony of the one or more detected colonies in the flow image tile. The identification can be performed using techniques identical or similar to those described with reference to block 608 in FIG. 6A. In some embodiments, at block 712, the system executes a plurality of processes in parallel on the system's GPU. In other words, the plurality of processes can be executed simultaneously. The plurality of processes corresponds to the plurality of detected sequencing colonies, respectively, and each process is executed to obtain the one or more properties (e.g., amplitude, location, profile) of the respective sequencing colony. In some embodiments, each process is an iterative process comprising a plurality of iterations, as described with reference to FIG. 6B.

Method 700 produces one or more properties for each detected colonies in the flow image tile. As discussed above, not all of the sequencing colonies captured in the flow image tile are detectable in block 704. Solely by way of example, in FIG. 5B, Detected Colony 1 may emit a relatively strong signal to be detected during the preamble flow step, but may not emit a strong enough signal to be detected in Flow Step 1. In some embodiments, the system still performs block 710 on Colony 1 even though it is not detected in block 702 (e.g., based on its location derived in preamble flow). Thus, with reference to FIG. 5A, at the end of process 520, the system can derive, for each sequencing colony in the catalog of sequencing colonies 510, the amplitude (and optionally other properties) for that flow step. Exemplary outputs of a flow step are provided in FIG. 5B.

It should be appreciated that all steps in all processes described herein can be performed using one or more GPUs using parallel processing. For example, each image can be processed simultaneously with another image; each image tile can be processed simultaneously with another time tile; each sequencing colony can be processed simultaneously with another sequencing colony in the same image tile; each pixel can be processed simultaneously with another pixel in the same image tile. For example, in a given image tile, the locations of multiple sequencing colonies can be detected and identified simultaneously.

Parallel processing significantly improves the throughput of the flow sequencing method. In one experiment, a flow sequencing method can involve hundreds of flow steps and each flow step can produce around one or more terabytes of image data. Embodiments of the present disclosure can process the image data at a high throughput (e.g., one or more gigabytes of image data per second). Further, the outputs are structured and stored in a memory-efficient manner. For example, for each flow, the system can store one or more bytes (e.g., 1 byte, 2 bytes, 4 bytes) of data for each sequencing colony's amplitude, one or more bytes (e.g., 1 byte, 2 bytes, 4 bytes) of data for each sequencing colony's location, and one or more bytes (e.g., 1 byte, 2 bytes, 4 bytes) of data for each sequencing colony's profile, in addition to a low-resolution background map and a low-resolution profile map as described herein. Thus, embodiments of the present disclosure improve the functioning of computer systems and sequencing platforms. Through novel data structures, processing logic, and use of GPUs, embodiments of the present disclosure provide improved memory usage, improved memory management, and improved processing to support the high-throughput requirement of the flow sequencing method to provide high-quality sequencing reads.

### Techniques for Improving Signal Detection of Denser Sequencing Colonies

Attaching a dense population of sequencing colonies on an open substrate of a sequencing platform (e.g., FIG. 3A) can be desirable to improve the efficiency of the flow sequencing method but can make detecting the sequencing colonies more difficult. The density of the sequencing colonies on a given substrate can be defined by a load ratio, which refers to the ratio between the number of sequencing colonies attached to the substrate and the maximum number of sequencing colonies that can be accommodated by the substrate (e.g., as defined by the maximum amount of space available for attachment of sequencing colonies). A higher load ratio indicates a denser population of sequencing colonies. In some flow sequencing methods, the load ratio can be around or over 90%. As the load ratio increases, it can be more difficult to detect the sequencing colonies because they are located closer to each other. The problem is further exacerbated when the profiles of the sequencing colonies become larger and/or when the amplitudes of the sequencing colonies are more varied. For example, a brighter sequencing colony can generate a strong crosstalk signal, which can make it more difficult to detect a nearby fainter sequencing colony.

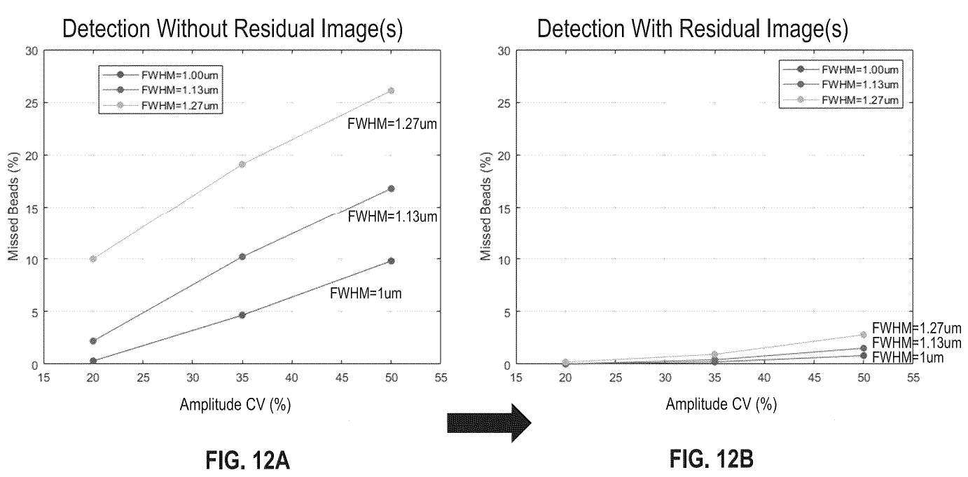

FIG. 12A illustrates how a larger sequencing colony profile and/or a larger amplitude variation among the sequencing colonies on a fairly dense surface (e.g., 90% load ratio) can negatively affect the performance of detection algorithms, in accordance with some embodiments. With reference to FIG. 12A, the x-axis corresponds to the coefficient of variation (“CV”) among the amplitudes of the sequencing colonies in a given image; the y-axis corresponds to the percentage of sequencing colonies missed by a detection algorithm (e.g., the algorithm described with reference to FIGS. 6A and 6B) in the image. As shown by each line, as the amplitude variation increases, a larger percentage of sequencing colonies is missed by the detection algorithm. Further, as shown by the three lines, as the profile (e.g., FWHM) of the sequencing colonies widens, a larger percentage of sequencing colonies is missed. The missed sequencing colonies can be especially problematic for a preamble flow step because the missed sequencing colonies would not be included in the catalog of sequencing colonies (e.g., 510 in FIG. 5A) and thus excluded from consideration in all subsequent flow steps. Further, the missing sequencing colonies can affect the accuracy of signal measurements or other properties in subsequent flow steps because the crosstalk signals generated by these missing sequencing colonies would not be accounted for.

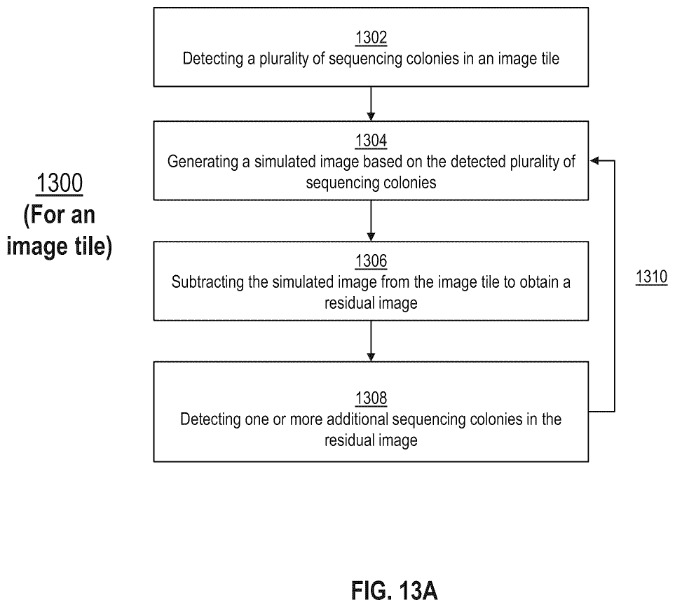

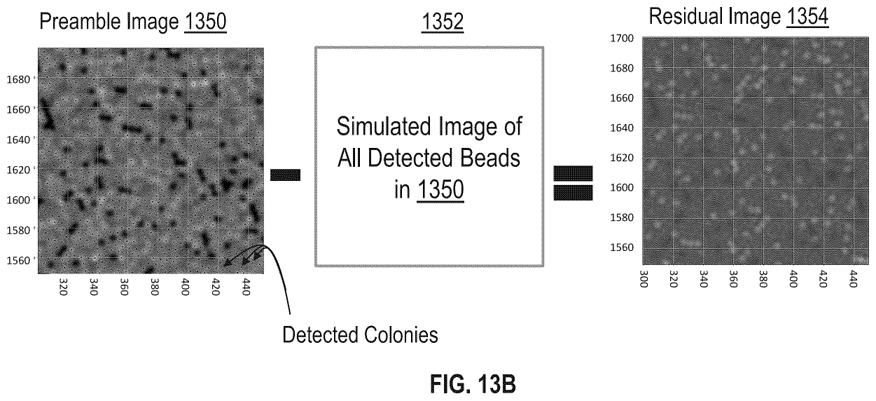

FIG. 13A illustrates an exemplary method 1300 for processing an image tile captured during flow sequencing, in accordance with some embodiments. In some embodiments, method 1300 is performed, for example, using one or more electronic devices implementing a software platform. In some examples, method 1300 is performed using a client-server system, and the blocks of method 1300 are divided up in any manner between the server and client device(s). In other examples, method 1300 is performed using only a client device or only multiple client devices. In method 1300, some blocks are, optionally, combined, the order of some blocks is, optionally, changed, and some blocks are, optionally, omitted. In some examples, additional steps may be performed in combination with the method 1300. Accordingly, the operations as illustrated (and described in greater detail below) are exemplary by nature and, as such, should not be viewed as limiting.

At block 1302, an exemplary system (e.g., one or more electronic devices) detects a plurality of sequencing colonies in the image tile. The image tile may be a reference image tile or a flow image tile. For example, the image tile may be a reference image tile, and the system can perform method 600 to detect the sequencing colonies in the image tile and determine one or more properties (e.g., amplitude, sub-pixel location, FWHM) of each detected sequencing colony. FIG. 13B illustrates an exemplary reference image tile 1350, with the dots indicating the detected sequencing colonies in the image tile. In some instances, a reference image tile 1350 may be from a preamble image (e.g., an image obtained during preamble sequencing flows, as described with respect to process 502).

At block 1304, the system generates a simulated image based on the detected plurality of sequencing colonies. The simulated image includes the detected plurality of sequencing colonies in block 1302. In some embodiments, each detected sequencing colony can be modeled in the simulated image using a profile model (e.g., pseudo-Voigt profile model) based on the amplitude and profile information (e.g., FWHM) of the sequencing colony determined in block 1302. Further, each detected sequencing colony is located in the simulated image at its corresponding location determined in block 1302. In some embodiments, the simulated image further includes background information determined in block 1302.

At block 1306, the system subtracts the simulated image from the image tile to obtain a residual image. FIG. 13B illustrates an exemplary residual image tile 1354. As shown, the residual image does not include the sequencing colonies detected in the original image 1350. Thus, the fainter sequencing colonies that were not detected in the original image 1350 appear more pronounced.

At block 1308, the system detects one or more additional sequencing colonies in the residual image. For example, the system can perform method 600 to detect sequencing colonies in the residual image and determine one or more properties (e.g., amplitude, sub-pixel location, FWHM) of each detected sequencing colony. If the image tile is a reference image tile, the additional sequencing colonies can be added to the catalog of sequencing colonies (e.g., catalog 510 in FIG. 5A).

In some embodiments, the system performs multiple iterations of blocks 1304-1308 to detect additional sequencing colonies. For example, in the second iteration, the system generates a new simulated image that includes the sequencing colonies detected in the previous iteration (i.e., using the residual image of the previous iteration) and subtracts the new simulated image from the residual image of the previous iteration to obtain a new residual image. Additional sequencing colonies can be then detected in the new residual image. If the image tile is a reference image tile, the additional sequencing colonies can be added to the catalog of sequencing colonies (510 in FIG. 5A).

In some embodiments, the system performs a predefined number of iterations of blocks 1304-1308. In some embodiments, after an iteration is performed, the system dynamically determines if another iteration is needed. The determination can be based on whether the total number of detected sequencing colonies exceeds a threshold (e.g., 95% of the total number of sequencing colonies captured in the image tile). Alternatively, the determination can be based on a comparison between the number of new sequencing colonies detected in the current iteration and the number of new sequencing colonies detected in the previous iteration. For example, the system can determine to forego another iteration if the sequencing colonies detected in the current iteration is less than 1% of the sequencing colonies detected in the previous iteration.

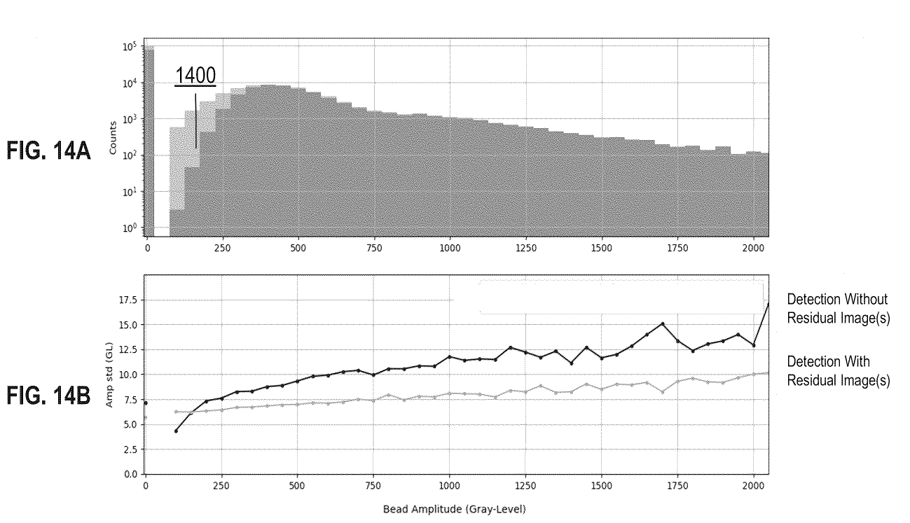

FIG. 12B illustrates how residual image(s) can improve the performance of detection algorithms, in accordance with some embodiments. As shown, the use of residual image(s) to detect sequencing colonies can reduce the percentage of missing sequencing colonies. FIG. 14A illustrates an exemplary histogram 1402 in which the x-axis represents the various sequencing colony amplitudes, and the y-axis represents the number of detected sequencing colonies having a given amplitude. The area 1400 represents the additional sequencing colonies detected by using residual images. As shown, the additional sequencing colonies have relatively low amplitudes and thus are missed when residual images are not used.

FIG. 14B illustrates that the use of residual image(s) can improve the measurement of signal amplitudes, in accordance with some embodiments. The x-axis represents the various sequencing colony amplitudes, and the y-axis represents the amplitude standard deviation of the sequencing colonies at a given amplitude range. As shown, detection with residual image(s) can lead to smaller amplitude deviations, suggesting more accurate amplitude measurements. This is because the use of residual image(s) can detect more sequencing colonies, and thus the crosstalk signals can be better estimated.

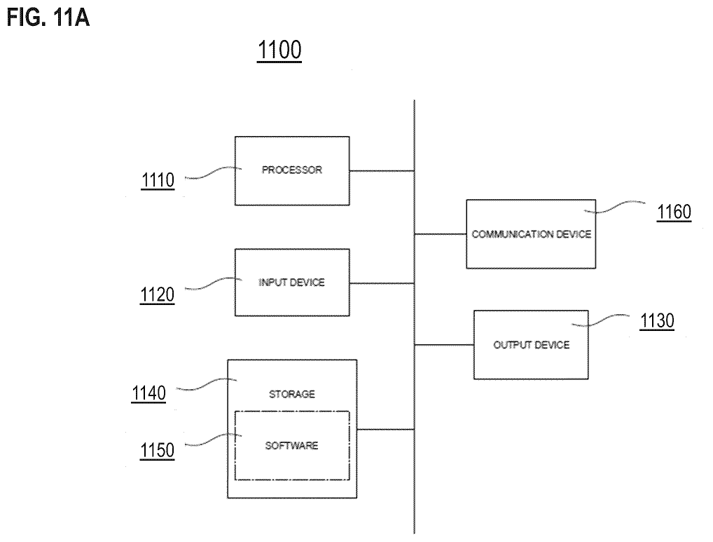

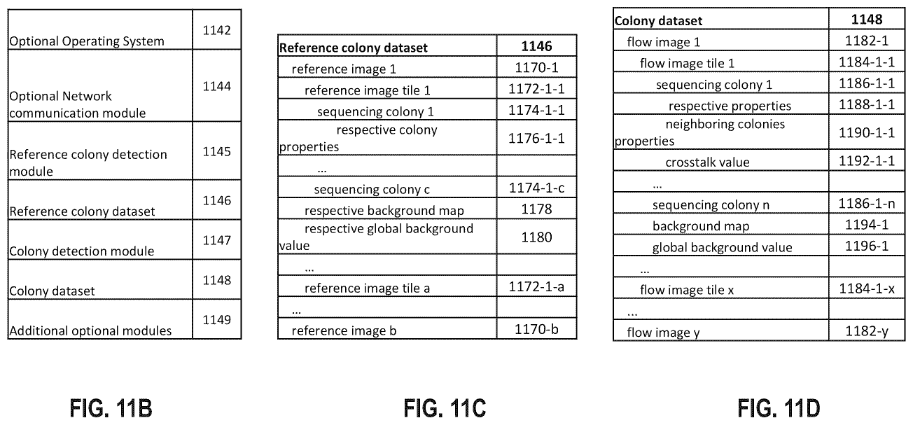

The operations described herein are optionally implemented by components depicted in FIG. 11A. FIG. 11A illustrates an example of a computing device 1100 in accordance with some instances. Device 1100 can be a host computer connected to a network. Device 1100 can be a client computer or a server. As shown in FIG. 11A, device 1100 can be any suitable type of microprocessor-based device, such as a personal computer, workstation, server, or handheld computing device (portable electronic device) such as a phone or tablet. The device 1100 can include, for example, one or more of processor 1110, input device 1120, output device 1130, storage 1140, and communication device 1160. Input device 1120 and output device 1130 can generally correspond to those described above and can either be connectable or integrated with the computer.

Input device 1120 can be any suitable device that provides input, such as a touch screen, keyboard or keypad, mouse, or voice-recognition device. Output device 1130 can be any suitable device that provides output, such as a touch screen, haptics device, or speaker.

Storage 1140 can be any suitable device that provides storage, such as an electrical, magnetic, or optical memory. In some instances, storage 1140 may comprise persistent memory, non-persistent memory, or a combination thereof (e.g., a device that includes both persistent and non-persistent memory). Non-persistent memory typically includes high-speed, random-access memory such as RAM and/or variations thereof. Storage 1140, especially persistent memory storage components, may optionally include one or more storage devices remotely located from processor(s) 1110. Persistent memory comprises anon-transitory computer-readable storage medium.

Communication device 1160 can include any suitable device capable of transmitting and receiving signals over a network, such as a network interface chip or device. The components of the computer can be connected in any suitable manner, such as via a physical bus or wirelessly.

Software 1150, which can be stored in storage 1140 (e.g., in persistent memory, non-persistent memory, or a combination thereof) and executed by processor 1110, can include, for example, the programming that embodies the functionality of the present disclosure (e.g., as embodied in the devices as described above). In some instances, software 1150 may comprise elements 1142, 1144, 1145, 1146, 1147, 1148, and 1149, specifically (e.g., as shown for example in FIGS. 11B, 11C, and 11D):
Optional Operating system 1142, which includes procedures for handling various basis system services and for performing hardware-dependent tasks;
Optional Network communication module (or instructions) 1144 for connecting computing device 1100 with other devices or with a communication network;
Reference colony detection module 1145 for identifying one or more colonies and their corresponding properties in reference images (e.g., using processes described herein with regards to FIG. 6A);
Reference colony dataset 1146, which includes, for each reference image 1170 in a plurality of reference images, for each reference image tile 1172 in a plurality of flow tiles, information corresponding to a plurality of sequencing colonies detected in the respective reference image flow tile, where this information includes, for each sequencing colony 1174 in the plurality of sequencing colonies, properties 1176 for the respective sequencing colony (e.g., initial location, amplitude, profile, etc.), and where information for each reference flow image 1170 further includes a respective background map 1178 and a respective global background value 1180;
Colony detection module 1147 for identifying one or more colonies and their corresponding properties in flow images (e.g., using processes described herein with regards to FIG. 6B and FIG. 7);
Colony dataset 1148, which includes, for each flow image 1182 in a plurality of flow images, for each flow image tile 1184 in a plurality of flow image tiles, information corresponding to a plurality of sequencing colonies detected in the respective flow image tile, where this information includes, for each sequencing colony 1186 in the plurality of sequencing colonies: (i) properties 1188 for the respective sequencing colony, (ii) properties 1190 for one or more colonies neighboring the respective sequencing colony, and (iii) a corresponding crosstalk value 1192 for the respective sequencing colony, and where information for each flow image 1182 further includes a respective background map 1194 and a respective global background value 1196; and
Optional additional modules 1149.

Software 1150 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1140, that can contain or store programming for use by or in connection with an instruction execution system, apparatus, or device.

Software 1150 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 1100 may be connected to a network (e.g., via optional network communication module 1144), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1100 can implement any operating system (e.g., optional operating system 1142) suitable for operating on the network. Software 1150 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

In some instances, one or more of the above-identified elements are stored in one or more of the previously mentioned storage devices and correspond to a set of instructions for performing a process as described herein. The above-identified modules, data, or programs (e.g., sets of instructions) need not be implemented separately; thus, various subsets of these modules, data, or programs may be combined or otherwise rearranged in various instances. In some instances, storage 1140 optionally stores a subset of the modules, data, and programs identified above. Furthermore, in some instances, storage 1140 stores additional modules, data, or programs not identified above.

Although FIG. 11A depicts a “computing device 1100,” the figure is intended more as functional description of the various features which may be present in computer systems for use with methods described herein than as a structural schematic of the implementations described herein. In practice, and as recognized by those of ordinary skill in the art, components shown separately could be combined and some components could be separated.

### EXAMPLES

### Example 1—Detection of Sequencing Colonies in Flow Sequencing

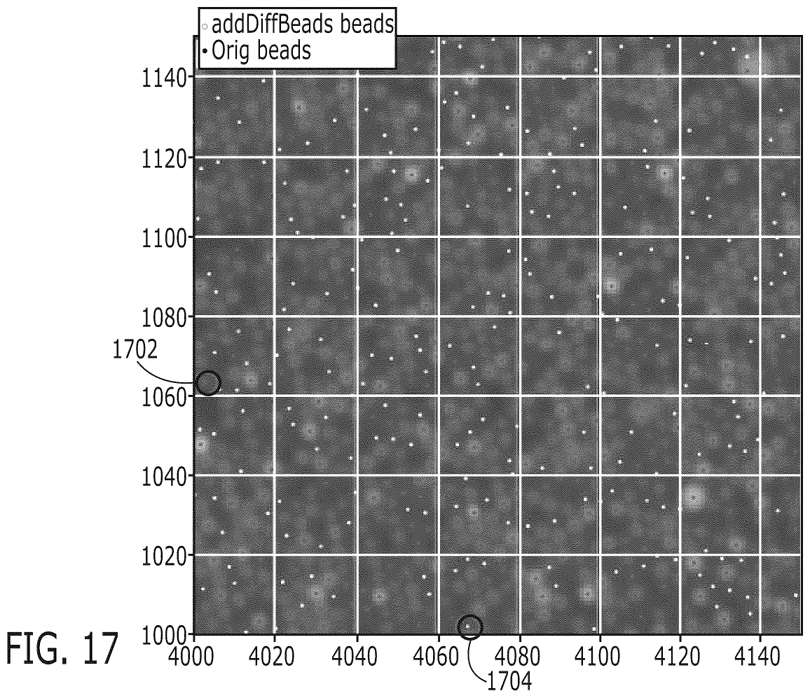

FIG. 17 provides an example of method 1300 (e.g., detecting additional sequencing colonies). This image was taken for a surface with a 1.4 um pitch (the average center-to-center distance between beads). An original detected bead 1702 (e.g., the initial set of detected sequencing colonies) is indicated. Additional beads are detected by the second detection iteration of method 1300 on the first flow image (reference flow), for example bead 1704. As can be seen in the image, a significant number of additional bead are detected by the additional detection iteration. This results in a corresponding increase in the amount of data that may be obtained from a single sequencing run, thus increasing the overall efficiency of the system.

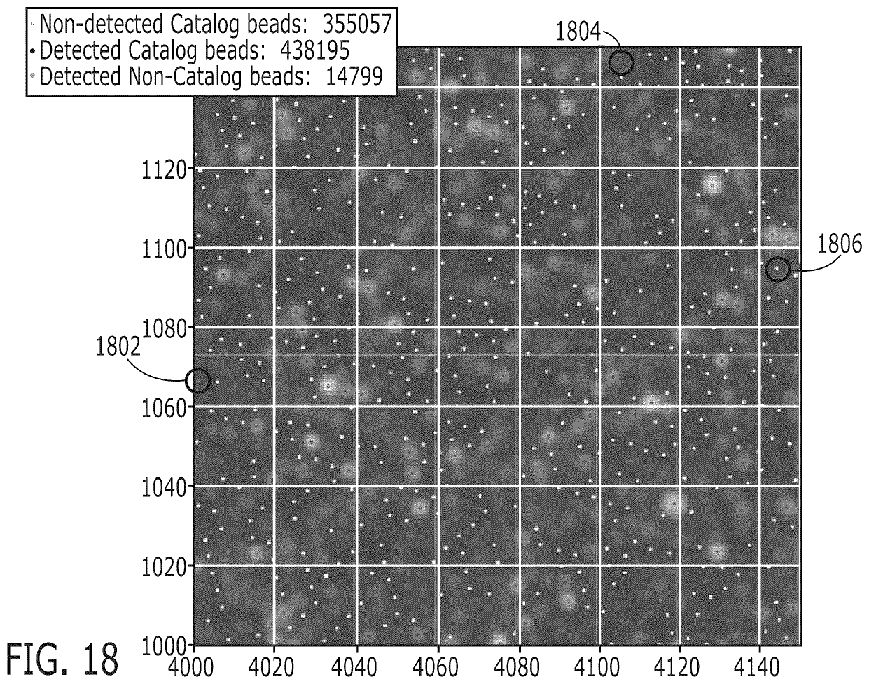

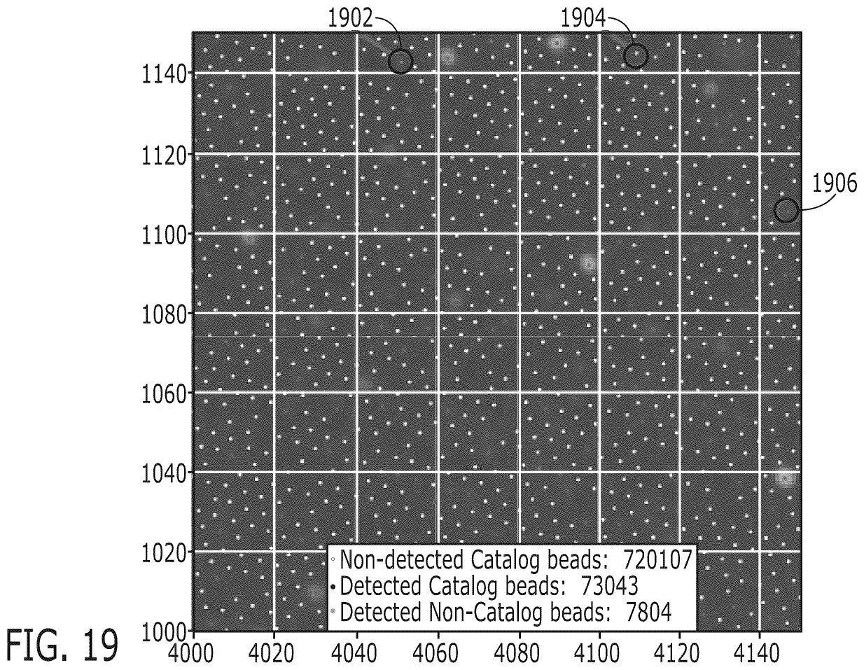

FIGS. 18 and 19 illustrate examples of detected sequencing colonies in a typical sequencing flow and in a zero-mer flow, respectively. These figures illustrate how some beads (e.g., sequencing colonies) that were not captured in the catalog process still may be detected in some flows. Likewise, some sequencing colonies that were cataloged may not be detected in every flow. In FIG. 18, for instance, three types of beads are indicated: non-detected catalog beads, (e.g., sequencing colonies that were cataloged—that is their locations are recorded—but were not detected in this individual sequencing flow), detected catalog beads (e.g., cataloged sequencing colonies that were detected in this sequencing flow), and detected non-catalog beads (e.g., sequencing colonies that were not cataloged—that is their locations were recoded as empty during the cataloging process or the beads changed location subsequent to the cataloging process). In the typical sequencing flow illustrated in FIG. 18, the undetected cataloged sequencing colonies (e.g., 1806) are about 44%, the detected cataloged sequencing colonies (1804) are about 54%, and non-cataloged but detected sequencing colonies (1802) are about 2% of the total detected and undetected sequencing colonies. In the zero-mer flow illustrated in FIG. 19, the undetected cataloged sequencing colonies (e.g., 1904) are about 90%, the detected cataloged sequencing colonies (1906) are about 10%, and non-cataloged but detected sequencing colonies (1902) are about 1% of the total detected and undetected sequencing colonies. In a zero-mer flow, by definition, cataloged sequencing colonies are expected to not be detected. In this image, the detected cataloged sequencing colonies are reference beads (e.g., beads that are always bright and are used to confirm the orientation of image tiles).

### Exemplary Embodiments

Among the provided embodiments are:
1. A method of determining nucleic acid sequences of a plurality of sequencing colonies, comprising:
   obtaining an input image of a surface, wherein the plurality of sequencing colonies is attached to the surface;
   detecting a set of sequencing colonies of the plurality of sequencing colonies in the input image;
   executing in parallel, using a graphics processor, a plurality of iterative processes to obtain signal amplitudes for the detected set of sequencing colonies,
      wherein each iterative process in the plurality of iterative processes corresponds to a respective sequencing colony in the detected set of sequencing colonies, and
      wherein each iterative process comprises:
         (a) obtaining amplitude, location, and profile estimates of one or more neighboring sequencing colonies that are adjacent to the respective sequencing colony;
         (b) calculating, using the graphics processor, a crosstalk value for the respective sequencing colony based on the amplitude, location, and profile estimates of the one or more neighboring sequencing colonies;
         (c) subtracting, using the graphics processor, the crosstalk value and a colony-specific background to obtain a current signal amplitude estimate of the respective sequencing colony;
         (d) performing a next iteration of (a)-(c) for a predetermined number of times or until a condition is met; and
   determining, at least partially based on the signal amplitudes for the detected set of sequencing colonies, portions of nucleic acid sequences of the plurality of sequencing colonies.
2. The method of embodiment 1, wherein each iterative process further comprises: determining, using the graphics processor, a current location estimate of the respective sequencing colony.
3. The method of any of embodiments 1-2, wherein each iterative process further comprises: determining, using the graphics processor, one or more current profile properties of the respective sequencing colony.
4. The method of any of embodiments 1-3, wherein the predetermined number of times is between 5-7 times.
5. The method of any of embodiments 1-4,
   wherein the input image is a first input image corresponding to a first flow step,
   wherein the obtained signal amplitudes correspond to the first flow step, and
   wherein the method further comprises:
      obtaining a second input image corresponding to a second flow step; and
      obtaining signal amplitudes corresponding to the second flow step.
6. The method of embodiment 5, further comprising: identifying, based on the signal amplitudes corresponding to the first flow step and the second flow step, the nucleic acid sequences of the plurality of sequencing colonies.
7. The method of any of embodiments 1-6, wherein the plurality of sequencing colonies is attached to a plurality of beads attached to the surface.
8. The method of any of embodiments 1-7, further comprising: capturing the input image of the surface.
9. The method of embodiment 8, further comprising: combining the plurality of sequencing colonies with nucleotides before capturing the input image, wherein at least a portion of the nucleotides are labeled.
10. The method of any of embodiments 1-9, wherein detecting the set of sequencing colonies comprises: applying one or more filters to the input image.
11. The method of embodiment 10, wherein the one or more filters comprise a Gaussian filter.
12. The method of embodiment 11, wherein the Gaussian filter is based on a known profile of a standard bead attached to the surface.
13. The method of embodiment 12, wherein the known profile includes a shape, a size, or a full-width at half-maximum value of the standard bead.
14. The method of embodiment 10, wherein the one or more filters comprise a low-pass filter and/or a high-pass filter.
15. The method of embodiment 10, further comprising: obtaining, based on a global background value, a binary image having a plurality of pixel values.
16. The method of embodiment 15, further comprising: grouping, based on the plurality of pixel values, pixels of the binary image into the detected set of sequencing colonies.
17. The method of embodiment 16, further comprising: determining a center pixel for each of the detected set of sequencing colonies.
18. The method of any of embodiments 1-17, further comprising determining an initial location for each of the detected set of sequencing colonies.
19. The method of embodiment 18, wherein the initial location is a sub-pixel location.
20. The method of embodiment 18, wherein the determination comprises a center of mass estimation.
21. The method of embodiment 19, further comprising: executing in parallel, using the graphics processor, a plurality of processes, each process corresponding to determining a respective sub-pixel location of a respective sequencing colony of the detected set of sequencing colonies.
22. The method of any of embodiments 1-21, further comprising: registering a center patch of the input image and a center patch of a reference image to obtain a horizontal shift and a vertical shift of the input image with respect to the reference image.
23. The method of embodiment 22, wherein the reference image is an image in which all captured sequencing colonies emit signals over a predefined threshold.
24. The method of embodiment 22, wherein the registering comprises:
   generating a first synthetic image corresponding to the center patch of the input image;
   generating a second synthetic image corresponding to the center patch of the reference image; and
   correlating the first synthetic image with the second synthetic image.
25. The method of embodiment 24, wherein each sequencing colony in the center patch of the input image is represented by the same Gaussian profile in the first synthetic image.
26. The method of embodiment 24, wherein each sequencing colony in the center patch of the reference image is represented by the same Gaussian profile in the second synthetic image.
27. The method of embodiment 24, wherein correlating the first synthetic image with the second synthetic image comprises performing a two-dimensional cross correlation using Fourier transform.
28. The method of embodiment 27, further comprising: generating an affine transformation between the reference image and the input image.
29. The method of embodiment 28, further comprising: iteratively refining one or more coefficients of the affine transformation.
30. The method of embodiment 29, further comprising:
   in each iteration:
      applying the affine transformation to the reference image;
      pairing one or more sequencing colonies in the input image with one or more transformed sequencing colonies in the reference image; and
      randomly selecting a number of paired sequencing colonies to refine the one or more coefficients of the affine transformation.
31. The method of any of embodiments 1-30, further comprising:
   dividing the input image into a plurality of sub-images;
   identifying, for each sub-image of the plurality of sub-images, a respective group of pixels in the respective sub-image based on pixel-specific amplitude information;
   extending, for each sub-image, the respective group of pixels;
   calculating, for each sub-image, a local background value based on the extended respective group of pixels; and
   generating a background map based on local background values of the plurality of sub-images.
32. The method of embodiment 31, further comprising: applying a mean filter to the background map.
33. The method of embodiment 31, further comprising: deriving a colony-specific background for each detected sequencing colony of the detected set of sequencing colonies by bi-linear interpolation of the background map.
34. The method of embodiment 31, further comprising: deriving a global background value based on a median of all extended groups of pixels for the plurality of sub-images.
35. The method of embodiment 3, wherein the one or more current profile properties include a current full width at half maximum (“FWHM”) estimate, a pseudo-Voigt Lorentzian weight (tail) parameter, or parameters of an elliptic model.
36. The method of embodiment 35, wherein the one or more current profile properties are determined based on an FWHM map.
37. The method of any of embodiments 1-36, wherein the surface is part of a substrate.
38. The method of any of embodiments 1-37, further comprising: capturing an arc-shaped or ring-shaped image of the surface.
39. The method of embodiment 38, further comprising: dividing the captured image into a plurality of image tiles, wherein the input image is one image tile of the plurality of image tiles.
40. The method of embodiment 39, further comprising: executing in parallel, using the graphics processor, a plurality of processes, each process corresponding to a respective image tile of the plurality of image tiles.
41. The method of any of embodiments 1-40, further comprising:
   detecting a plurality of sequencing colonies in a reference image;
   generating a simulated image based on the plurality of detected sequencing colonies in the reference image;
   subtracting the simulated image from the reference image to obtain a residual image; and
   detecting one or more additional sequencing colonies based on the residual image.
42. A system of determining nucleic acid sequences of a plurality of sequencing colonies, comprising:
   one or more processors;
   a memory; and
   one or more programs, wherein the one or more programs are stored in the memory and configured to be executed by the one or more processors, the one or more programs including instructions for:
   obtaining an input image of a surface, wherein the plurality of sequencing colonies is attached to the surface;
   detecting a set of sequencing colonies of the plurality of sequencing colonies in the input image;
   executing in parallel, using a graphics processor, a plurality of iterative processes to obtain signal amplitudes for the detected set of sequencing colonies,
      wherein each iterative process in the plurality of iterative processes corresponds to a respective sequencing colony in the detected set of sequencing colonies, and
      wherein each iterative process comprises:
         (a) obtaining amplitude, location, and profile estimates of one or more neighboring sequencing colonies that are adjacent to the respective sequencing colony;
         (b) calculating, using the graphics processor, a crosstalk value for the respective sequencing colony based on the amplitude, location, and profile estimates of the one or more neighboring sequencing colonies;
         (c) subtracting, using the graphics processor, the crosstalk value and a colony-specific background to obtain a current signal amplitude estimate of the respective sequencing colony;
         (d) performing a next iteration of (a)-(c) for a predetermined number of times or until a condition is met; and
   determining, at least partially based on the signal amplitudes for the detected set of sequencing colonies, portions of nucleic acid sequences of the plurality of sequencing colonies.
43. The system of embodiment 42, wherein each iterative process further comprises: determining, using the graphics processor, a current location estimate of the respective sequencing colony.
44. The system of any of embodiments 42-43, wherein each iterative process further comprises: determining, using the graphics processor, one or more current profile properties of the respective sequencing colony.
45. The system of any of embodiments 42-44, wherein the predetermined number of times is between 5-7 times.
46. The system of any of embodiments 42-45,
   wherein the input image is a first input image corresponding to a first flow step,
   wherein the obtained signal amplitudes correspond to the first flow step, and
   wherein the one or more programs further comprise instructions for:
      obtaining a second input image corresponding to a second flow step; and
      obtaining signal amplitudes corresponding to the second flow step.
47. The system of embodiment 46, wherein the one or more programs further include instructions for: identifying, based on the signal amplitudes corresponding to the first flow step and the second flow step, the nucleic acid sequences of the plurality of sequencing colonies.
48. The system of any of embodiments 42-47, wherein the plurality of sequencing colonies is attached to a plurality of beads attached to the surface.
49. The system of any of embodiments 42-48, wherein the one or more programs further include instructions for: capturing the input image of the surface.
50. The system of embodiment 49, wherein the one or more programs further include instructions for: combining the plurality of sequencing colonies with nucleotides before capturing the input image, wherein at least a portion of the nucleotides are labeled.
51. The system of any of embodiments 42-50, wherein detecting the set of sequencing colonies comprises: applying one or more filters to the input image.
52. The system of embodiment 51, wherein the one or more filters comprise a Gaussian filter.
53. The system of embodiment 52, wherein the Gaussian filter is based on a known profile of a standard bead attached to the surface.
54. The system of embodiment 53, wherein the known profile includes a shape, a size, or a full-width at half-maximum value of the standard bead.
55. The system of embodiment 51, wherein the one or more filters comprise a low-pass filter and/or a high-pass filter.
56. The system of embodiment 51, wherein the one or more programs further include instructions for: obtaining, based on a global background value, a binary image having a plurality of pixel values.
57. The system of embodiment 56, wherein the one or more programs further include instructions for: grouping, based on the plurality of pixel values, pixels of the binary image into the detected set of sequencing colonies.
58. The system of embodiment 57, wherein the one or more programs further include instructions for: determining a center pixel for each of the detected set of sequencing colonies.
59. The system of any of embodiments 42-58, wherein the one or more programs further include instructions for determining an initial location for each of the detected set of sequencing colonies.
60. The system of embodiment 59, wherein the initial location is a sub-pixel location.
61. The system of embodiment 59, wherein the determination comprises a center of mass estimation.
62. The system of embodiment 60, wherein the one or more programs further include instructions for: executing in parallel, using the graphics processor, a plurality of processes, each process corresponding to determining a respective sub-pixel location of a respective sequencing colony of the detected set of sequencing colonies.
63. The system of any of embodiments 42-62, wherein the one or more programs further include instructions for: registering a center patch of the input image and a center patch of a reference image to obtain a horizontal shift and a vertical shift of the input image with respect to the reference image.
64. The system of embodiment 63, wherein the reference image is an image in which all captured sequencing colonies emit signals over a predefined threshold.
65. The system of embodiment 63, wherein the registering comprises:
   generating a first synthetic image corresponding to the center patch of the input image;
   generating a second synthetic image corresponding to the center patch of the reference image; and
   correlating the first synthetic image with the second synthetic image.
66. The system of embodiment 65, wherein each sequencing colony in the center patch of the input image is represented by the same Gaussian profile in the first synthetic image.
67. The system of embodiment 65, wherein each sequencing colony in the center patch of the reference image is represented by the same Gaussian profile in the second synthetic image.
68. The system of embodiment 65, wherein correlating the first synthetic image with the second synthetic image comprises performing a two-dimensional cross correlation using Fourier transform.
69. The system of embodiment 68, wherein the one or more programs further include instructions for: generating an affine transformation between the reference image and the input image.
70. The system of embodiment 69, wherein the one or more programs further include instructions for: iteratively refining one or more coefficients of the affine transformation.
71. The system of embodiment 70, wherein the one or more programs further include instructions for:
   in each iteration:
      applying the affine transformation to the reference image;
      pairing one or more sequencing colonies in the input image with one or more transformed sequencing colonies in the reference image; and
      randomly selecting a number of paired sequencing colonies to refine the one or more coefficients of the affine transformation.
72. The system of any of embodiments 42-71, wherein the one or more programs further include instructions for:
   dividing the input image into a plurality of sub-images;
   identifying, for each sub-image of the plurality of sub-images, a respective group of pixels in the respective sub-image based on pixel-specific amplitude information;
   extending, for each sub-image, the respective group of pixels;
   calculating, for each sub-image, a local background value based on the extended respective group of pixels; and
   generating a background map based on local background values of the plurality of sub-images.
73. The system of embodiment 72, wherein the one or more programs further include instructions for: applying a mean filter to the background map.
74. The system of embodiment 72, wherein the one or more programs further include instructions for: deriving a colony-specific background for each detected sequencing colony of the detected set of sequencing colonies by bi-linear interpolation of the background map.
75. The system of embodiment 72, wherein the one or more programs further include instructions for: deriving a global background value based on a median of all extended groups of pixels for the plurality of sub-images.
76. The system of embodiment 44, wherein the one or more current profile properties include a current full width at half maximum (“FWHM”) estimate, a pseudo-Voigt Lorentzian weight (tail) parameter, or parameters of an elliptic model.
77. The system of embodiment 76, wherein the one or more current profile properties are determined based on an FWHM map.
78. The system of any of embodiments 42-77, wherein the surface is part of a substrate.
79. The system of any of embodiments 42-78, wherein the one or more programs further include instructions for: capturing an arc-shaped or ring-shaped image of the surface.
80. The system of embodiment 79, wherein the one or more programs further include instructions for: dividing the captured image into a plurality of image tiles, wherein the input image is one image tile of the plurality of image tiles.
81. The system of embodiment 80, wherein the one or more programs further include instructions for: executing in parallel, using the graphics processor, a plurality of processes, each process corresponding to a respective image tile of the plurality of image tiles.
82. The system of any of embodiments 42-81, wherein the one or more programs further include instructions for:
   detecting a plurality of sequencing colonies in a reference image;
   generating a simulated image based on the plurality of detected sequencing colonies in the reference image;
   subtracting the simulated image from the reference image to obtain a residual image; and
   detecting one or more additional sequencing colonies based on the residual image.
83. A non-transitory computer-readable storage medium storing one or more programs for determining nucleic acid sequences of a plurality of sequencing colonies, the one or more programs comprising instructions, which when executed by one or more processors of one or more electronic devices, cause the electronic devices to:
   obtain an input image of a surface, wherein the plurality of sequencing colonies is attached to the surface;
   detect a set of sequencing colonies of the plurality of sequencing colonies in the input image;
   execute in parallel, using a graphics processor, a plurality of iterative processes to obtain signal amplitudes for the detected set of sequencing colonies,
      wherein each iterative process in the plurality of iterative processes corresponds to a respective sequencing colony in the detected set of sequencing colonies, and
      wherein each iterative process comprises:
         (a) obtaining amplitude, location, and profile estimates of one or more neighboring sequencing colonies that are adjacent to the respective sequencing colony;
         (b) calculating, using the graphics processor, a crosstalk value for the respective sequencing colony based on the amplitude, location, and profile estimates of the one or more neighboring sequencing colonies;
         (c) subtracting, using the graphics processor, the crosstalk value and a colony-specific background to obtain a current signal amplitude estimate of the respective sequencing colony;
         (d) performing a next iteration of (a)-(c) for a predetermined number of times or until a condition is met; and determine, at least partially based on the signal amplitudes for the detected set of sequencing colonies, portions of nucleic acid sequences of the plurality of sequencing colonies.
84. The non-transitory computer-readable storage medium of embodiment 83, wherein each iterative process further comprises: determining, using the graphics processor, a current location estimate of the respective sequencing colony.
85. The non-transitory computer-readable storage medium of any of embodiments 83-84, wherein each iterative process further comprises: determining, using the graphics processor, one or more current profile properties of the respective sequencing colony.
86. The non-transitory computer-readable storage medium of any of embodiments 83-85, wherein the predetermined number of times is between 5-7 times.
87. The non-transitory computer-readable storage medium of any of embodiments 83-86,
   wherein the input image is a first input image corresponding to a first flow step,
   wherein the obtained signal amplitudes correspond to the first flow step, and
   wherein the one or more programs further comprise instructions for:
      obtaining a second input image corresponding to a second flow step; and
      obtaining signal amplitudes corresponding to the second flow step.
88. The non-transitory computer-readable storage medium of embodiment 87, wherein the one or more programs further comprise instructions for: identifying, based on the signal amplitudes corresponding to the first flow step and the second flow step, the nucleic acid sequences of the plurality of sequencing colonies.
89. The non-transitory computer-readable storage medium of any of embodiments 83-88, wherein the plurality of sequencing colonies is attached to a plurality of beads attached to the surface.
90. The non-transitory computer-readable storage medium of any of embodiments 83-89, wherein the one or more programs further comprise instructions for: capturing the input image of the surface.
91. The non-transitory computer-readable storage medium of embodiment 90, wherein the one or more programs further comprise instructions for: combining the plurality of sequencing colonies with nucleotides before capturing the input image, wherein at least a portion of the nucleotides are labeled.
92. The non-transitory computer-readable storage medium of any of embodiments 83-91, wherein detecting the set of sequencing colonies comprises: applying one or more filters to the input image.
93. The non-transitory computer-readable storage medium of embodiment 92, wherein the one or more filters comprise a Gaussian filter.
94. The non-transitory computer-readable storage medium of embodiment 93, wherein the Gaussian filter is based on a known profile of a standard bead attached to the surface.
95. The non-transitory computer-readable storage medium of embodiment 94, wherein the known profile includes a shape, a size, or a full-width at half-maximum value of the standard bead.
96. The non-transitory computer-readable storage medium of embodiment 92, wherein the one or more filters comprise a low-pass filter and/or a high-pass filter.
97. The non-transitory computer-readable storage medium of embodiment 92, wherein the one or more programs further comprise instructions for: obtaining, based on a global background value, a binary image having a plurality of pixel values.
98. The non-transitory computer-readable storage medium of embodiment 97, wherein the one or more programs further comprise instructions for: grouping, based on the plurality of pixel values, pixels of the binary image into the detected set of sequencing colonies.
99. The non-transitory computer-readable storage medium of embodiment 98, wherein the one or more programs further comprise instructions for: determining a center pixel for each of the detected set of sequencing colonies.
100. The non-transitory computer-readable storage medium of any of embodiments 83-99, wherein the one or more programs further comprise instructions for determining an initial location for each of the detected set of sequencing colonies.
101. The non-transitory computer-readable storage medium of embodiment 100, wherein the initial location is a sub-pixel location.
102. The non-transitory computer-readable storage medium of embodiment 100, wherein the determination comprises a center of mass estimation.
103. The non-transitory computer-readable storage medium of embodiment 101, wherein the one or more programs further comprise instructions for: executing in parallel, using the graphics processor, a plurality of processes, each process corresponding to determining a respective sub-pixel location of a respective sequencing colony of the detected set of sequencing colonies.
104. The non-transitory computer-readable storage medium of any of embodiments 83-103, wherein the one or more programs further comprise instructions for: registering a center patch of the input image and a center patch of a reference image to obtain a horizontal shift and a vertical shift of the input image with respect to the reference image.
105. The non-transitory computer-readable storage medium of embodiment 104, wherein the reference image is an image in which all captured sequencing colonies emit signals over a predefined threshold.
106. The non-transitory computer-readable storage medium of embodiment 104, wherein the registering comprises:
   generating a first synthetic image corresponding to the center patch of the input image;
   generating a second synthetic image corresponding to the center patch of the reference image; and
   correlating the first synthetic image with the second synthetic image.
107. The non-transitory computer-readable storage medium of embodiment 106, wherein each sequencing colony in the center patch of the input image is represented by the same Gaussian profile in the first synthetic image.
108. The non-transitory computer-readable storage medium of embodiment 106, wherein each sequencing colony in the center patch of the reference image is represented by the same Gaussian profile in the second synthetic image.
109. The non-transitory computer-readable storage medium of embodiment 106, wherein correlating the first synthetic image with the second synthetic image comprises performing a two-dimensional cross correlation using Fourier transform.
110. The non-transitory computer-readable storage medium of embodiment 109, wherein the one or more programs further comprise instructions for: generating an affine transformation between the reference image and the input image.
111. The non-transitory computer-readable storage medium of embodiment 110, wherein the one or more programs further comprise instructions for: iteratively refining one or more coefficients of the affine transformation.
112. The non-transitory computer-readable storage medium of embodiment 111, wherein the one or more programs further comprise instructions for:
   in each iteration:
      applying the affine transformation to the reference image;
      pairing one or more sequencing colonies in the input image with one or more transformed sequencing colonies in the reference image; and
      randomly selecting a number of paired sequencing colonies to refine the one or more coefficients of the affine transformation.
113. The non-transitory computer-readable storage medium of any of embodiments 83-112, wherein the one or more programs further comprise instructions for:
   dividing the input image into a plurality of sub-images;
   identifying, for each sub-image of the plurality of sub-images, a respective group of pixels in the respective sub-image based on pixel-specific amplitude information;
   extending, for each sub-image, the respective group of pixels;
   calculating, for each sub-image, a local background value based on the extended respective group of pixels; and
   generating a background map based on local background values of the plurality of sub-images.
114. The non-transitory computer-readable storage medium of embodiment 113, wherein the one or more programs further comprise instructions for: applying a mean filter to the background map.
115. The non-transitory computer-readable storage medium of embodiment 113, wherein the one or more programs further comprise instructions for: deriving a colony-specific background for each detected sequencing colony of the detected set of sequencing colonies by bi-linear interpolation of the background map.
116. The non-transitory computer-readable storage medium of embodiment 113, wherein the one or more programs further comprise instructions for: deriving a global background value based on a median of all extended groups of pixels for the plurality of sub-images.
117. The non-transitory computer-readable storage medium of embodiment 85, wherein the one or more current profile properties include a current full width at half maximum (“FWHM”) estimate, a pseudo-Voigt Lorentzian weight (tail) parameter, or parameters of an elliptic model.
118. The non-transitory computer-readable storage medium of embodiment 117, wherein the one or more current profile properties are determined based on an FWHM map.
119. The non-transitory computer-readable storage medium of any of embodiments 83-118, wherein the surface is part of a substrate.
120. The non-transitory computer-readable storage medium of any of embodiments 83-119, wherein the one or more programs further comprise instructions for: capturing an arc-shaped or ring-shaped image of the surface.
121. The non-transitory computer-readable storage medium of embodiment 120, wherein the one or more programs further comprise instructions for: dividing the captured image into a plurality of image tiles, wherein the input image is one image tile of the plurality of image tiles.
122. The non-transitory computer-readable storage medium of embodiment 121, wherein the one or more programs further comprise instructions for: executing in parallel, using the graphics processor, a plurality of processes, each process corresponding to a respective image tile of the plurality of image tiles.
123. The non-transitory computer-readable storage medium of any of embodiments 83-122, wherein the one or more programs further comprise instructions for:
   detecting a plurality of sequencing colonies in a reference image;
   generating a simulated image based on the plurality of detected sequencing colonies in the reference image;
   subtracting the simulated image from the reference image to obtain a residual image; and
   detecting one or more additional sequencing colonies based on the residual image.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

1.-123. (canceled)

124. A method of determining nucleic acid sequences of a plurality of sequencing colonies, comprising:
obtaining an input image of a surface, wherein the plurality of sequencing colonies are attached to the surface;
detecting a set of sequencing colonies of the plurality of sequencing colonies in the input image;
executing in parallel, using a graphics processor, a plurality of iterative processes to obtain signal amplitudes for the detected set of sequencing colonies,
wherein each iterative process in the plurality of iterative processes corresponds to a respective sequencing colony in the detected set of sequencing colonies, and
wherein each iterative process comprises:
(a) obtaining amplitude, location, and profile estimates of one or more neighboring sequencing colonies that are adjacent to the respective sequencing colony;
(b) calculating, using the graphics processor, a crosstalk value for the respective sequencing colony based on the amplitude, location, and profile estimates of the one or more neighboring sequencing colonies;
(c) subtracting, using the graphics processor, the crosstalk value and a colony-specific background to obtain a current signal amplitude estimate of the respective sequencing colony;
(d) performing a next iteration of (a)-(c) for a predetermined number of times or until a condition is met; and
determining, at least partially based on the signal amplitudes for the detected set of sequencing colonies, portions of nucleic acid sequences of the plurality of sequencing colonies.

125. The method of claim 124, wherein each iterative process further comprises: determining, using the graphics processor, a current location estimate of the respective sequencing colony.

126. The method of claim 124, wherein each iterative process further comprises: determining, using the graphics processor, one or more current profile properties of the respective sequencing colony.

127. The method of claim 126, wherein the one or more current profile properties include a current full width at half maximum (“FWHM”) estimate, a pseudo-Voigt Lorentzian weight (tail) parameter, or parameters of an elliptic model.

128. The method of claim 124,
wherein the input image is a first input image corresponding to a first flow step,
wherein the obtained signal amplitudes correspond to the first flow step, and
wherein the method further comprises:
obtaining a second input image corresponding to a second flow step; and
obtaining signal amplitudes corresponding to the second flow step.

129. The method of claim 128, further comprising: identifying, based on the signal amplitudes corresponding to the first flow step and the second flow step, the nucleic acid sequences of the plurality of sequencing colonies.

130. The method of claim 124, further comprising:
a. capturing the input image of the surface; and
b. combining the plurality of sequencing colonies with nucleotides before capturing the input image, wherein at least a portion of the nucleotides are labeled.

131. The method of claim 124, wherein detecting the set of sequencing colonies comprises: applying one or more filters to the input image.

132. The method of claim 131, wherein the one or more filters comprise a Gaussian filter.

133. The method of claim 131, further comprising: obtaining, based on a global background value, a binary image having a plurality of pixel values.

134. The method of claim 133, further comprising: grouping, based on the plurality of pixel values, pixels of the binary image into the detected set of sequencing colonies.

135. The method of claim 124, further comprising determining an initial location for each of the detected set of sequencing colonies.

136. The method of claim 135, wherein the initial location is a sub-pixel location.

137. The method of claim 135, wherein the determination comprises a center of mass estimation.

138. The method of claim 124, further comprising:
dividing the input image into a plurality of sub-images;
identifying, for each sub-image of the plurality of sub-images, a respective group of pixels in the respective sub-image based on pixel-specific amplitude information;
extending, for each sub-image, the respective group of pixels;
calculating, for each sub-image, a local background value based on the extended respective group of pixels; and
generating a background map based on local background values of the plurality of sub-images.

139. The method of claim 138, further comprising: deriving a colony-specific background for each detected sequencing colony of the detected set of sequencing colonies by bi-linear interpolation of the background map.

140. The method of claim 138, further comprising: deriving a global background value based on a median of all extended groups of pixels for the plurality of sub-images.

141. The method of claim 124, further comprising: capturing an arc-shaped or ring-shaped image of the surface.

142. The method of claim 141, further comprising: dividing the captured image into a plurality of image tiles, wherein the input image is one image tile of the plurality of image tiles.

143. The method of claim 124, further comprising:
detecting a plurality of sequencing colonies in a reference image;
generating a simulated image based on the plurality of detected sequencing colonies in the reference image;
subtracting the simulated image from the reference image to obtain a residual image; and
detecting one or more additional sequencing colonies based on the residual image.
